(19) United States
(12) Patent Application Publication
(10) Pub. No.: US 2023/0193355 A1
(43) Pub. Date: Jun. 22, 2023

(54) METHODS AND COMPOSITIONS FOR HIGH-THROUGHPUT TARGET SEQUENCING IN SINGLE CELLS

(71) Applicant: SINGLERON (NANJING) BIOTECHNOLOGIES, LTD., Nanjing (CN)

(72) Inventors: Nan Fang, Nanjing (CN); Wenqi Zhu, Nanjing (CN); Xiuheng Ding, Nanjing (CN)

(21) Appl. No.: 17/996,195

(22) PCT Filed: Apr. 15, 2021
(86) PCT No.: PCT/CN2021/087517

§ 371 Date: Oct. 13, 2022

(30) Foreign Application Priority Data
Apr. 16, 2020 (WO) PCT/CN2020/085185
Apr. 28, 2020 (WO) PCT/CN2020/087525
Apr. 6, 2021 (WO) PCT/CN2021/085610

(51) Int. Cl. C12Q 1/6806

(52) U.S. Cl. CPC C12Q 1/6806

(57) ABSTRACT

Provided include methods, compositions and kits for single cell target sequencing, including but not limited to, high-throughput detection of nucleic acid sequences of single cell T cell receptor, high-throughput detection of expressed viral sequences in host cells, detection of cancer druggable mutations (e.g., lung cancer druggable mutations) in single cells, and simultaneous detection of targeted regions and whole transcriptome in single cells.

### CROSS-REFERENCE TO RELATED APPLICATIONS

This application claims the benefit of priority to PCT Application Nos. PCT/CN2020/085185, filed on Apr. 16, 2020, PCT/CN2020/087525, filed on Apr. 28, 2020, PCT/CN2021/085610, filed on Apr. 6, 2021; the content of these related applications is incorporated herein by reference in its entirety.

### REFERENCE TO SEQUENCE LISTING

The present application is being filed along with a Sequence Listing in electronic format. The Sequence Listing is provided as a file entitled Sequence_Listing_76PP-328946-WO, created Apr. 13, 2021, which is 29 kilobytes in size. The information in the electronic format of the Sequence Listing is incorporated herein by reference in its entirety.

### BACKGROUND

### Field

The present application generally relates to molecular biology. More specifically, provided herein include methods, compositions, kits and systems for high-throughput single cell target sequencing.

### Description of the Related Art

Single-cell transcriptome technology has been rapidly developed. However, current technology cannot fully reveal the integrity and complexity of the transcriptome expression profile.

### SUMMARY

Provided include methods, compositions and kits for single cell target sequencing, including but not limited to, high-throughput detection of nucleic acid sequences of single cell T cell receptor, high-throughput detection of expressed viral sequences in host cells, detection of cancer druggable mutations (e.g., lung cancer druggable mutations) in single cells, and simultaneous detection of targeted regions and whole transcriptome in single cells.

Disclosed herein include methods for single cell analysis. In some embodiments, a method for single cell analysis comprises partitioning a cell and a bead attached with a plurality of barcode oligonucleotides into a partition. Each barcode oligonucleotide of the plurality of barcode oligonucleotides can comprise a cell barcode and a unique molecular identifier (UMI). First barcode oligonucleotides of the plurality of barcode oligonucleotides each can comprise a poly-dT sequence capable of binding to a poly-A tail of a first messenger ribonucleic acid (mRNA) target. Second barcode oligonucleotides of the plurality of barcode oligonucleotides each can comprise a poly-dT sequence and a probe sequence. The probe sequence is not a poly-dT sequence. The probe sequence can be capable of binding to a second RNA target at a sequence that is not a poly-A sequence. The method can comprise hybridizing the first barcode oligonucleotides and the second barcode oligonucleotides attached to the bead in the partition with RNA targets associated with the cell in the partition. The method can comprise reverse transcribing the RNA targets hybridized to the first barcode oligonucleotides and the second barcode oligonucleotides to generate barcoded complementary deoxyribonucleic acids (cDNAs). The method can comprise amplifying the barcoded cDNAs. The method can comprise analyzing the amplified barcoded cDNAs, or products thereof.

In some embodiments, analyzing the amplified barcoded cDNAs comprises sequencing the amplified barcoded cDNAs to obtain sequencing information. In some embodiments, analyzing the amplified barcoded cDNAs comprises determining an expression profile of each of one or more the RNA targets using a number of UMIs with different sequences associated with the RNA target in the sequencing information. Analyzing the amplified barcoded cDNAs can comprise determining an expression profile of the second RNA target using a number of UMIs with different sequences associated with the second RNA target in the sequencing information. The expression profile can comprise an absolute abundance or a relative abundance. In some embodiments, analyzing the amplified barcoded cDNAs comprises determining a number of amplified barcoded cDNAs of each of one or more the RNA targets comprising UMIs with different sequences. Analyzing the amplified barcoded cDNAs can comprise determining a number of amplified barcoded cDNAs the second RNA target comprising UMIs with different sequences. Analyzing the amplified barcoded cDNAs can comprise determining sequences of the amplified barcoded cDNAs of the second RNA target, or a portion thereof, comprising UMIs with different sequences.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode, (ii) a unique molecular identifier (UMI), and (iiia) a poly-dT sequence and/or (iiib) a probe sequence. The poly-dT sequence can be capable of binding to a poly-A region of a first nucleic acid target. The probe sequence is not a poly-dT sequence. The probe sequence can be capable of binding to a second nucleic acid target. The method can comprise barcoding nucleic acid targets associated with the cell in each partition of the partitions using first barcode oligonucleotides and second barcode oligonucleotides attached to the bead in the partition to generate barcoded nucleic acids. The method can comprise sequencing the barcoded nucleic acids, or products thereof, to obtain sequencing information.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode and (ii) a unique molecular identifier (UMI). The method can comprise barcoding nucleic acid targets associated with the cell in each partition of the partitions to generate barcoded nucleic acids using (a) extension primers and/or a probe sequence and (b) the first barcode oligonucleotides and the second barcode oligonucleotides attached to the bead in the partition. The poly-dT sequence can be capable of binding to a poly-A region of a first nucleic acid target. The probe sequence is not a poly-dT sequence. The probe sequence can be capable of binding to a second nucleic acid target. The first barcode oligonucleotides and the second barcode oligonucleotides attached to the bead in the partition can be used as template switching oligonucleotides for barcoding the nucleic acid targets. The method can comprise sequencing the barcoded nucleic acids, or products thereof, to obtain sequencing information.

In some embodiments, the nucleic acid targets comprise ribonucleic acids (RNAs), messenger RNAs (mRNAs), and/or deoxyribonucleic acids (DNAs). The nucleic acid targets can comprise nucleic acid targets of the cell, from the cell, in the cell (which can be released from the cell after cell lysis), and/or on the surface of the cell.

In some embodiments, the method comprises releasing the nucleic acids form the cell prior to barcoding the nucleic acid targets associated with the cell. The method comprises lysing the cell to release the nucleic acids form the cell.

In some embodiments, barcoding the nucleic acids associated with the cell comprises hybridizing the first barcode oligonucleotides and the second barcode oligonucleotides attached to the bead in each partition of the partitions with nucleic acid targets associated with the cell in the partition. Barcoding the nucleic acids associated with the cell can comprise extending the first barcode oligonucleotides and the second barcode oligonucleotides attached to the bead and hybridized to the nucleic acid targets using the nucleic acids as templates to generate single-stranded barcoded nucleic acids. Barcoding the nucleic acids associated with the cell can comprise generating double-stranded barcoded nucleic acids from the single-stranded barcoded nucleic acids. Extending the single-stranded barcoded nucleic acids comprises further extending the single-stranded barcoded nucleic acids using a template switching oligonucleotide.

In some embodiments, the method comprises pooling the beads prior to extending the first barcode oligonucleotides and the second barcode oligonucleotides. The method can comprise pooling the beads prior to generating the double-stranded barcoded nucleic acids. In some embodiments, extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in bulk. Generating the double-stranded barcoded nucleic acids can comprise generating the double-stranded barcoded nucleic acids from the single-stranded barcoded nucleic acids in bulk. In some embodiments, the method comprises pooling the beads subsequent to extending the first barcode oligonucleotides and the second barcode oligonucleotides to generate the single-stranded barcoded nucleic acids. The method can comprise pooling the beads subsequent to generating the double-stranded barcoded nucleic acids. In some embodiments, extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in the partition. Generating the double-stranded barcoded nucleic acids can comprise generating the double-stranded barcoded nucleic acids from the single-stranded barcoded nucleic acids in the partition.

In some embodiments, the method comprises amplifying the barcoded nucleic acid to generate amplified barcoded nucleic acids. Amplifying the barcoded nucleic acids can comprise amplifying the barcoded nucleic acids using polymerase chain reaction (PCR) to generate the amplified barcoded nucleic acids. The method can comprise processing the amplified barcoded nucleic acids to generate processed barcoded nucleic acids. Sequencing the barcoded nucleic acids can comprise sequencing the processed barcoded nucleic acids.

In some embodiments, processing the amplified barcoded nucleic acids comprises fragmenting the amplified barcoded nucleic acids to generate fragmented barcoded nucleic acids. Fragmenting the amplified barcoded nucleic acids can comprise fragmenting the amplified barcoded nucleic acids enzymatically to generate the fragmented barcoded nucleic acids. Processing the amplified barcoded nucleic acids can comprise adding a second polymerase chain reaction (PCR) primer-binding sequence. The second PCR primer-binding sequence can comprise a Read 2 sequence. Processing the amplified barcoded nucleic acids comprises generating processed barcoded nucleic acids comprising sequencing primer sequences from the fragmented barcoded nucleic acids. The sequencing primer sequences can comprise a P5 sequence and a P7 sequence.

In some embodiments, the method comprises analyzing the sequencing information. In some embodiments, analyzing the sequencing information comprises determining an expression profile of each of one or more nucleic acid targets of the nucleic acid targets associated with the cell using a number of UMIs with different sequences associated with the nucleic acid target in the sequencing information. Analyzing the sequencing information can comprise determining an expression profile of the second nucleic acid target using a number of UMIs with different sequences associated with the second nucleic acid target in the sequencing information. Analyzing the sequencing information can comprise determining sequences of the second nucleic acid target, or a portion thereof, associated with UMIs with different sequences. The expression profile can comprise an absolute abundance or a relative abundance. The expression profile can comprise an RNA expression profile, an mRNA expression profile, and/or a protein expression profile.

In some embodiments, sequencing the barcoded nucleic acids, or products thereof, comprises sequencing products of the barcoded nucleic acids each comprising a P5 sequence, a Read 1 sequence, a cell barcode, a UMI, a poly-dT sequence, a probe sequence, a sequence of a nucleic acid target or a portion thereof, a Read 2 sequence, a sample index, and/or a P7 sequence to obtain sequencing information.

In some embodiments, the partition is a droplet or a microwell. The plurality of partitions can comprise a plurality of microwells of a microwell array. The plurality of partitions can comprise at least 1000 partitions.

In some embodiments, at least 50% of partitions of the plurality of partitions comprise a single cell of the plurality of cells and a single bead of the plurality of beads. At most 10% of partitions of the plurality of partitions can comprise two or more cells of the plurality of cells. At most 10% of partitions of the plurality of partitions can comprise no cell of the plurality of cells. At most 10% of partitions of the plurality of partitions can comprise two or more beads of the plurality of beads. At most 10% of partitions of the plurality of partitions can comprise no bead of the plurality of beads.

In some embodiments, a length of the poly-dT sequence is at least 10 nucleotides in length. The probe sequence can be at least 10 nucleotides in length. In some embodiments, first barcode oligonucleotides of the plurality of barcode oligonucleotides each comprises a poly-dT sequence. The poly-dT sequence can be capable of binding to a poly-A region of a first nucleic acid target. In some embodiments, the poly-dT sequences of the first barcode oligonucleotides of the plurality of barcode oligonucleotides attached to a bead of the beads are identical. The poly-dT sequences of the first barcode oligonucleotides attached to the beads can be identical.

In some embodiments, second barcode oligonucleotides of the plurality of barcode oligonucleotides each comprises a probe sequence. The probe sequence is not a poly-dT sequence. The probe sequence can be capable of binding to a second nucleic acid target.

In some embodiments, second barcode oligonucleotides of the plurality of barcode oligonucleotides each comprises a poly-dT sequence and a probe sequence. The probe sequence is not a poly-dT sequence. The probe sequence can be capable of binding to a second nucleic acid target. In some embodiments, second barcode oligonucleotides of the plurality of barcode oligonucleotides comprise probe sequences that are not poly-dT sequences. The probe sequences can be capable of binding to an identical second nucleic acid target. In some embodiments, second barcode oligonucleotides of the plurality of barcode oligonucleotides comprise probe sequences that are not poly-dT sequences. The probe sequences can be capable of binding to different second nucleic acid targets.

In some embodiments, the probe sequences of barcode oligonucleotides of the plurality of barcode oligonucleotides comprise a degenerate sequence. A length of the degenerate sequence can be at least 3. The degenerate sequence can span, or correspond to, a mutation. In some embodiments, the probe sequences of barcode oligonucleotides of the plurality of barcode oligonucleotides span a region of interest. In some embodiments, wherein the probe sequence is adjacent a region of interest.

The region of interest can comprise a variable region of a T-cell receptor (TCR). The TCR can be TCR alpha or TCR beta. In some embodiments, the region of interest comprises a mutation. In some embodiments, the mutation comprises an insertion, a deletion, or a substitution. The substitution can comprise a single-nucleotide variant (SNV) or a single-nucleotide polymorphism (SNP). The mutation can be related to a cancer.

In some embodiments, the cell barcodes of two barcode oligonucleotides of the plurality of barcode oligonucleotides attached to a bead of the beads comprise an identical sequence. The cell barcodes of two barcode oligonucleotides attached to two beads of the beads can comprise different sequences. The cell barcode of each barcode oligonucleotide can be at least 6 nucleotides in length.

In some embodiments, the UMIs of two barcode oligonucleotides attached to a bead of the beads can comprise different sequences. The UMIs of two barcode oligonucleotides attached to two beads of the beads can comprise an identical sequence. The UMI of each barcode oligonucleotide can be at least 6 nucleotides in length.

In some embodiments, each barcode oligonucleotide of the plurality of barcode oligonucleotides comprises a first polymerase chain reaction (PCR) primer-binding sequence. The first PCR primer-binding sequence can comprise a Read 1 sequence.

In some embodiments, barcode oligonucleotides of the plurality of barcode oligonucleotides are reversibly attached to, covalently attached to, or irreversibly attached to the bead. In some embodiments, the bead is a gel bead. The gel bead can be degradable upon application of a stimulus. The stimulus can comprise a thermal stimulus, a chemical stimulus, a biological stimulus, a photo-stimulus, or a combination thereof. In some embodiments, the bead is a solid bead. The bead can be a magnetic bead.

In some embodiments, the number of different second nucleic acid targets is at least 10. In some embodiments, the second nucleic acid target comprises a T-cell receptor (TCR), or an RNA (e.g., mRNA) product thereof. The probe sequence can be capable of binding to a constant region, or a portion thereof, of the TCR. The TCR can be TCR alpha or TCR beta. In some embodiments, the cell is a cancer cell. The second nucleic acid target is a cancer gene, or an RNA (e.g., mRNA) product thereof.

In some embodiments, the cell is infected with a virus. The second nucleic acid target is a gene of the virus, or a nucleic acid product (e.g., RNA) thereof. The virus can be an RNA virus. The second nucleic acid target can comprise an RNA of the gene of the virus. The method can thus determine a transcriptomic profile of the cell and a nucleic acid (e.g., RNA) profile of the virus.

In some embodiments, the second nucleic acid target comprises no poly-A tail and/or no poly-A region. In some embodiments, the second nucleic acid target comprises a poly-A region. The poly-A region can be a poly-A tail.

In some embodiments, an abundance of molecules of the second nucleic acid target hybridized to (or barcoded using) the second barcode oligonucleotides is higher than an abundance of molecules of the second nucleic acid target hybridized to (or barcoded using) the first barcode oligonucleotides. The method can thus enrich the second nucleic acid target.

In some embodiments, the abundance of the molecules of the second nucleic acid target comprises a number of occurrences of the molecules of the second nucleic acid target. In some embodiments, the abundance of the molecules of the second nucleic acid target can comprise a number of occurrences of the molecules of the second nucleic acid target relative to a number of the first barcode oligonucleotides or a number of the second barcode oligonucleotides.

In some embodiments, the method comprises enriching the one or more second nucleic acid targets using one or more enrichment primers. Enriching the second nucleic acid targets comprises enriching the second nucleic acid targets using the enrichment primers of a panel. The panel can be a customizable panel.

Disclosed herein include compositions for single cell sequencing or single cell analysis. In some embodiments, a composition for single cell sequencing or single cell analysis comprises a plurality of beads of the present disclosure. The cell barcodes of the plurality of barcode oligonucleotides attached to each of the plurality of beads can be identical. The cell barcodes of barcodes oligonucleotide attached to different beads of the plurality of beads can be different. The plurality of beads can comprise at least 100 beads.

Disclosed herein include kits for single cell sequencing or single cell analysis. In some embodiments, a kit for single cell sequencing or single cell analysis comprises a composition comprising a plurality of beads of the present disclosure. The kit can comprise instructions of using the composition for single cell sequencing or single cell analysis.

Disclosed herein includes methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode, a unique molecular identifier (UMI), and a poly-dT sequence. The method can comprise adding, to 3′-end of each of barcode oligonucleotides of the plurality of barcode oligonucleotides, a probe sequence that is a not poly-dT sequence and is capable of binding to a nucleic acid target.

In some embodiments, adding the probe sequence comprises adding the probe sequence to the 3′-end of each of the barcode oligonucleotides of the plurality of barcode oligonucleotides chemically. In some embodiments, adding the probe sequence comprises adding the probe sequence to the 3′-end of each of the barcode oligonucleotides of the plurality of barcode oligonucleotides using an enzyme. In some embodiments, the enzyme is a ligase. Adding the probe sequence can comprise ligating a probe oligonucleotide comprising the probe sequence to the 3′-end of each of the barcode oligonucleotides of the plurality of barcode oligonucleotides using the ligase. In some embodiments, the enzyme is a DNA polymerase. Adding the probe sequence can comprise synthesizing the probe sequence at the 3′-end of each of the barcode oligonucleotides of the plurality of barcode oligonucleotides using the DNA polymerase.

Disclosed herein include methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode and a unique molecular identifier (UMI). The method can comprise adding to 3′-end of each of barcode oligonucleotides of the plurality of barcode oligonucleotides (i) a poly-dT sequence and/or (ii) a probe sequence that is a non-poly-dT sequence and is capable of binding to a nucleic acid target.

Disclosed herein include a method for analyzing TCR sequence at single cell level. The method can, for example, comprises: (a) capturing the RNA from a single cell with an oligo-dT primer combined with probe sequence that binding to TCR RNA sequence; (b) reversing transcribe the RNA to cDNA with the oligo-dT primer and TCR-recognizing sequence; (c) amplifying cDNA; (d) amplifying TCR sequence; and (e) analyzing amplified cDNA. In some embodiments, the primer sequence additionally comprises a sequence that acts as cell barcode that identifies each single cells; a sequence that can be used as PCR primer-binding sequence for amplification of the cDNA.

In some embodiments, the primer sequence comprises a unique molecular index (UMI) sequence that can be used to quantify cDNA. In some embodiments, the probe sequence is added by using an enzyme. In some embodiments, the probe sequence is added chemically. In some embodiments, the enzyme is a ligase, to add specific sequence to the 3′ of oligo-dT. In some embodiments, the enzyme is a DNA polymerase, to add specific sequence to the 3′ of PolyT. In some embodiments, the target enrichment method is PCR. In some embodiments, the PCR used in Target Enrichment is annealing to TCR variable Region. In some embodiments, the analysis method is sequencing.

Disclosed herein includes a method for analyzing virus sequence, at single cell level. The method can, for example, comprises: (a) capturing the RNA from a single cell with an oligo-dT primer combined with probe sequence that binding to viral RNA sequence; (b) reversing transcribe the RNA to cDNA with the oligo-dT primer and virus-recognizing sequence; (c) amplifying cDNA; and (d) analyzing amplified cDNA.

In some embodiments, the primer sequence additionally comprises a sequence that acts as cell barcode that identifies each single cell; a sequence that can be used as PCR primer-binding sequence for amplification of the cDNA. In some embodiments, the primer sequence comprises a unique molecular index (UMI) sequence that can be used to quantify cDNA. In some embodiments, the probe sequence is added by using an enzyme. In some embodiments, the probe sequence is added chemically. In some embodiments, the enzyme is a ligase, to add specific sequence to the magnetic capture bead. In some embodiments, the enzyme is a DNA polymerase, to add specific sequence to magnetic capture bead. In some embodiments, the viral RNA sequence can be derived from any RNA virus. In some embodiments, the analysis method is sequencing.

Disclosed herein include a method for analyzing targeted regions at single cell level. The method can, for example, comprises: (a) capturing the RNA from a single cell with an oligo-dT primer combined with probe sequence that binding to targeted sequence; (b) reverse transcribing the RNA to cDNA with the oligo-dT primer and targeted specific primer; (c) amplifying cDNA; (d) analyzing the amplified cDNA; and (e) enriching the target sequence with specific primers.

In some embodiments, the oligo-dT primer sequence additionally comprises a sequence that acts as cell barcode that identifies each single cells; a sequence that can be used as PCR primer-binding sequence for amplification of the cDNA. In some embodiments, the oligo-dT primer sequence comprise a unique molecular index (UMI) sequence that can be used to quantify cDNA. In some embodiments, the probe sequence is added by using an enzyme. In some embodiments, the probe sequence is added chemically. In some embodiments, the enzyme is a ligase to add specific sequence to the magnetic capture bead. In some embodiments, the enzyme is a DNA polymerase to add specific sequence to magnetic capture bead. In some embodiments, the target sequence can be derived from any RNA. In some embodiments, the analysis method is sequencing. In some embodiments, the target genes will be enriched by customized panel.

### BRIEF DESCRIPTION OF DRAWINGS

FIG. 1 is a schematic diagram showing a non-limiting workflow for capturing mRNA and TCR sequence. Panel (a) shows RNA capture and reverse transcription, panel (b) shows cDNA amplification, panel (c) shows gene expression library construction, and panel (d) shows TCR target-enrichment.

FIG. 2 is a schematic diagram showing a non-limiting embodiment in which a cell barcoding capture magnetic bead is used to capture mRNA and TCR sequence.

FIG. 3 is an amplified cDNA map.

FIG. 4 is a TCR target enrichment 1 map.

FIG. 5 is a TCR target enrichment 2 map.

FIG. 6 is a TCR library map.

FIGS. 7A-B are plots showing scRNA-seq results.

FIGS. 8A-B are graphs showing detection of TCR sequences in two human oral cancer samples.

FIGS. 9A-D are plots showing TCR sequencing results.

FIG. 10 is a schematic diagram showing a non-limiting workflow for capturing mRNA and viral RNA. Panel (a) shows cell lysis and capture of host mRNA and viral RNA, panel (b) shows reverse transcription, and panel (c) shows cDNA amplification and library construction,

FIG. 11 is a schematic diagram showing a non-limiting embodiment in which a cell barcoding capture magnetic bead is used to capture host mRNA and Viral RNA. Panel (a) shows composition of the cell barcoding capture magnetic bead, and panel (b) shows single cells partition and cell barcoding bead loading.

FIG. 12 shows sequence of synthetic SARS-COV-2 RNA

FIG. 13 shows the portion of sequence read assigned to host gene and viral genome.

FIG. 14 shows the cell number contain different rate of viral read.

FIG. 15 shows sorting of cells by the expression of COVID-19.

FIGS. 16A-B are plots showing viral sequencing results.

FIG. 17 is a schematic diagram showing a non-limiting example of the cell barcoding bead.

FIG. 18 is a visualization of EGFR gene T790M mutation.

FIG. 19 shows t-SNE plots. The clusters (left) and the detected mutation (right) of NCI-H1975 were captured by magnetic beads, which containing polyT and gene specific probes.

FIG. 20 shows t-SNE plots. The clusters (left) and the detected viruses (right) of NCI-H1975 were captured by magnetic beads, which only containing polyT probes.

FIG. 21A shows raw read summary, FIG. 21B shows mapping summary, and FIG. 21C shows important quota.

FIG. 22 is a graph showing cell summary.

FIGS. 23A-B are plots showing sequencing results using druggable S beads to analyze A549/U937 cells.

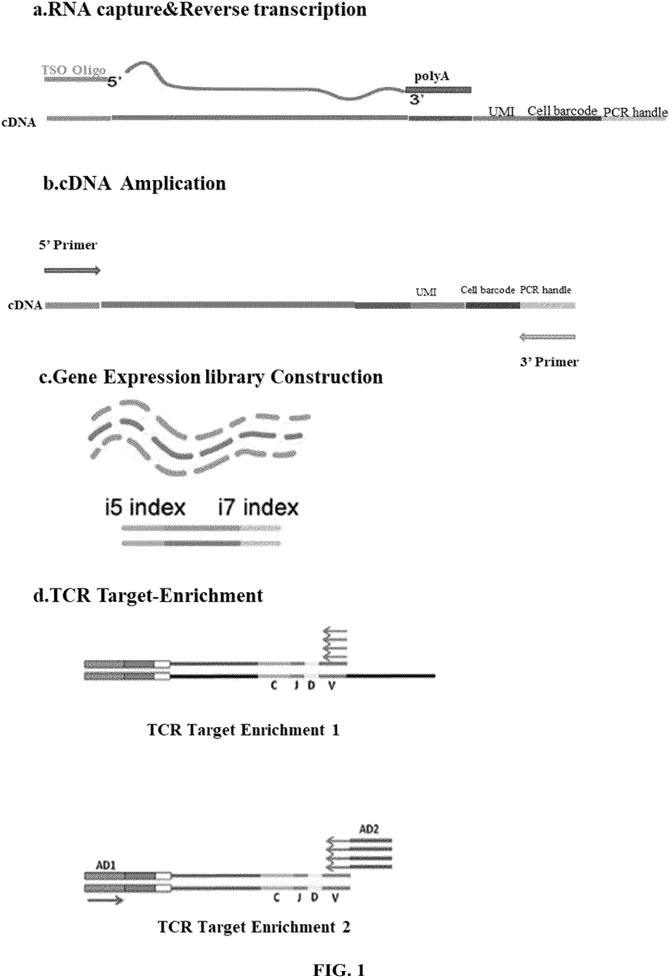

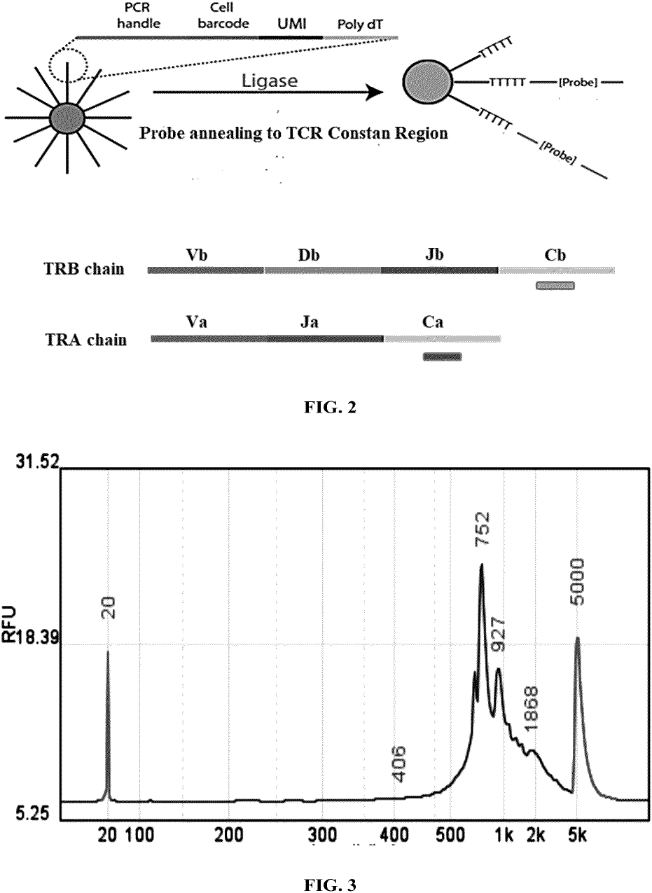

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Provided include methods, compositions and kits for single cell target sequencing, including but not limited to, high-throughput detection of nucleic acid sequences of single cell T cell receptor, high-throughput detection of expressed viral sequences in host cells, detection of cancer druggable mutations (e.g., lung cancer druggable mutations) in single cells, and simultaneous detection of targeted regions and whole transcriptome in single cells.

Disclosed herein include methods for single cell analysis. In some embodiments, a method for single cell analysis comprises partitioning a cell and a bead attached with a plurality of barcode oligonucleotides into a partition. Each barcode oligonucleotide of the plurality of barcode oligonucleotides can comprise a cell barcode and a unique molecular identifier (UMI). First barcode oligonucleotides of the plurality of barcode oligonucleotides each can comprise a poly-dT sequence capable of binding to a poly-A tail of a first messenger ribonucleic acid (mRNA) target. Second barcode oligonucleotides of the plurality of barcode oligonucleotides each can comprise a poly-dT sequence and a probe sequence. The probe sequence is not a poly-dT sequence. The probe sequence can be capable of binding to a second RNA target at a sequence that is not a poly-A sequence. The method can comprise hybridizing the first barcode oligonucleotides and the second barcode oligonucleotides attached to the bead in the partition with RNA targets associated with the cell in the partition. The method can comprise reverse transcribing the RNA targets hybridized to the first barcode oligonucleotides and the second barcode oligonucleotides to generate barcoded complementary deoxyribonucleic acids (cDNAs). The method can comprise amplifying the barcoded cDNAs. The method can comprise analyzing the amplified barcoded cDNAs, or products thereof.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode, (ii) a unique molecular identifier (UMI), and (iiia) a poly-dT sequence and/or (iiib) a probe sequence. The poly-dT sequence can be capable of binding to a poly-A region of a first nucleic acid target. The probe sequence is not a poly-dT sequence. The probe sequence can be capable of binding to a second nucleic acid target. The method can comprise barcoding nucleic acid targets associated with the cell in each partition of the partitions using first barcode oligonucleotides and second barcode oligonucleotides attached to the bead in the partition to generate barcoded nucleic acids. The method can comprise sequencing the barcoded nucleic acids, or products thereof, to obtain sequencing information.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode and (ii) a unique molecular identifier (UMI). The method can comprise barcoding nucleic acid targets associated with the cell in each partition of the partitions to generate barcoded nucleic acids using (a) extension primers and/or a probe sequence and (b) the first barcode oligonucleotides and the second barcode oligonucleotides attached to the bead in the partition. The poly-dT sequence can be capable of binding to a poly-A region of a first nucleic acid target. The probe sequence is not a poly-dT sequence. The probe sequence can be capable of binding to a second nucleic acid target. The first barcode oligonucleotides and the second barcode oligonucleotides attached to the bead in the partition can be used as template switching oligonucleotides for barcoding the nucleic acid targets. The method can comprise sequencing the barcoded nucleic acids, or products thereof, to obtain sequencing information.

Disclosed herein include compositions for single cell sequencing or single cell analysis. In some embodiments, a composition for single cell sequencing or single cell analysis comprises a plurality of beads of the present disclosure. The cell barcodes of the plurality of barcode oligonucleotides attached to each of the plurality of beads can be identical. The cell barcodes of barcodes oligonucleotide attached to different beads of the plurality of beads can be different. The plurality of beads can comprise at least 100 beads.

Disclosed herein include kits for single cell sequencing or single cell analysis. In some embodiments, a kit for single cell sequencing or single cell analysis comprises a composition comprising a plurality of beads of the present disclosure. The kit can comprise instructions of using the composition for single cell sequencing or single cell analysis.

Disclosed herein includes methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode, a unique molecular identifier (UMI), and a poly-dT sequence. The method can comprise adding, to 3′-end of each of barcode oligonucleotides of the plurality of barcode oligonucleotides, a probe sequence that is a not poly-dT sequence and is capable of binding to a nucleic acid target.

Disclosed herein include methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode and a unique molecular identifier (UMI). The method can comprise adding to 3′-end of each of barcode oligonucleotides of the plurality of barcode oligonucleotides (i) a poly-dT sequence and/or (ii) a probe sequence that is a non-poly-dT sequence and is capable of binding to a nucleic acid target.

Disclosed herein include a method for analyzing TCR sequence at single cell level. The method can, for example, comprises: (a) capturing the RNA from a single cell with an oligo-dT primer combined with probe sequence that binding to TCR RNA sequence; (b) reversing transcribe the RNA to cDNA with the oligo-dT primer and TCR-recognizing sequence; (c) amplifying cDNA; (d) amplifying TCR sequence; and (e) analyzing amplified cDNA. In some embodiments, the primer sequence additionally comprises a sequence that acts as cell barcode that identifies each single cells; a sequence that can be used as PCR primer-binding sequence for amplification of the cDNA.

Disclosed herein includes a method for analyzing virus sequence, at single cell level. The method can, for example, comprises: (a) capturing the RNA from a single cell with an oligo-dT primer combined with probe sequence that binding to viral RNA sequence; (b) reversing transcribe the RNA to cDNA with the oligo-dT primer and virus-recognizing sequence; (c) amplifying cDNA; and (d) analyzing amplified cDNA.

Disclosed herein include a method for analyzing targeted regions at single cell level. The method can, for example, comprises: (a) capturing the RNA from a single cell with an oligo-dT primer combined with probe sequence that binding to targeted sequence; (b) reverse transcribing the RNA to cDNA with the oligo-dT primer and targeted specific primer; (c) amplifying cDNA; (d) analyzing the amplified cDNA; and (e) enriching the target sequence with specific primers.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

### Detection of Sequences of T Cell Receptors

B lymphocytes and T lymphocytes participate in the acquired immune responses. Human T cells develop in the thymus from progenitors originating in hematopoietic tissues. During their development, T cells acquire the ability to recognize foreign antigens and provide protection against many different types of pathogens. This functional flexibility is facilitated by the expression of highly polymorphic surface receptors called T cell receptors (TCRs). The diversity of TCRs, B cell receptors (BCRs) and secreted antibodies constitutes the core of a complex immune system and serves as a key defense component that protects the body from invasion by viral, bacteria and other foreign substances. TCR is a heterodimer--αβ chain (~ 95%, TRA, TRB) or γδ chain (~ 5%). Each chain can be divided into variable and constant domains. Each peptide chain can be divided into variable region (V region), constant region (C region), transmembrane region and cytoplasmic region. The variable region of α chain is encoded by V and J gene fragments. The variable region of the β chain is encoded by three gene segments: V, D, and J. The V regions (Vα, Vβ) of the two peptide chains, α and β, have three hypervariable regions: CDR1, CDR2, and CDR3, of which the CDR3 region (also called hypervariable region) has the largest variation, which directly determines the antigen binding specificity of TCR.

Due to the rearrangement of the V (D) J gene and the random deletion of germline nucleotides, the TCR profiles are extremely diverse. In humans, it is theoretically estimated that the diversity of TCR-αβ receptors exceeds 1012 in the thymus, and the diversity directly determines the antigen binding specificity of TCR.

In recent years, due to the advances of gene sequencing technologies, high-throughput sequencing technology (such as RNA-Seq) has been used to detect the diversity of immune receptors, and Immune repertoire sequencing can be applied to the fields of vaccine and pharmaceutical research and development, discovery of biomarkers, detection of Minimal Residual Disease (MRD), research of autoimmune diseases and post-transplant monitoring. For example in the study of disease-specific biomarkers, disease-specific CDR3 can be found in people with the same disease through high-throughput sequencing. After verification, these CDR3 sequences can be used as Biomarker representing the disease and can be found in peripheral blood; Research on autoimmune diseases such as rheumatoid arthritis, can identify potential autologous clones by high-throughput sequencing to quantify the T cell repertoire of peripheral blood of early or diagnosed rheumatoid arthritis, as a basis for the early diagnosis of medication. It can promote the development of vaccines for different populations by analyzing the effects of people of different ages after injection of vaccines. For tumor research, disease guidance can be monitored by comparing changes in the immune repertoire of patients before and after medication to prevent tumor recurrence.

However, the traditional RNA-seq measures the average expression level of tissue samples or cell populations, which makes the difference between cells likely masked by the average value, and cannot specifically describe the diversity of lymphocytes or clonetypes that constitute the immune response. On the other hand, bulk RNA-seq cannot determine which TCRA and TCRB chains combine to form a specific TCR, which is essential for many functional and therapeutic applications. Therefore, the establishment of a method for detecting the diversity of TCR at single cell level is particularly important for promoting the application of immune receptors sequencing in early clinical diagnosis, efficacy evaluation, and prognosis judgment.

At present, there are several methods and reagents for single cell immune receptors detection, such as SMARTer Human scTCR a / b Profiling Kit from Takara/Clontech, through sorting single cells by manual or flow cytometry into 96-well PCR Plate, each well is an independent reaction, through the processes of cell lysis, reverse transcription, and PCR amplification, the enrichment of immune receptor sequences was achieved. However, the disadvantages of Clontech are as follows: Clontech generally relies on plate- or well-based microfluidics and is therefore limited in the number of cells that can be processed, typically 10-100. Additionally, a large number of sequencing reads are generally required to computationally reconstruct paired antigen receptors. As such, the cost per cell is relatively high, estimated at $50-$100 USD.

Chromium Single Cell V (D) J Reagent Kits launched by 10X Genomics has greatly improved the detection throughput compared to Clontech’s products. By encapsulating single cells and hydrogel beads containing cell barcode in individual droplets, TCR from thousands of single cells can be processed and then detected in parallel. However, the disadvantages of Clontech are as follows: The mapping rate of TCR sequencing is relatively low , the Median UMI detection value of TCR a chain is relatively low resulting the low detection rate of TCR a chain.

Disclosed herein include methods, compositions, kits and systems for high-throughput detection of TCR sequences at single cell level. Probe binding to TCR sequence can be combined with oligo-dT to capture mRNA, improving the capture efficiency of TCR sequences. For example, the probe and oligo-dT contain the same PCR handle sequence, so that TCR can be amplified by multiplex PCR. Optionally, the probe and oligo-dT can be combined with a oligonucleotide sequence that can act as cell barcode to distinguish each single cell from other cells, so that thousands or more of single cells can be analyzed in parallel. This method can also be used in combination with a microfluidic system where each cell in a sample can be partitioned to individual micro-chambers. Single cells can be lyzed in the micro-chambers, and mRNA and TCR sequences can be captured at the same time.

Compositions (e.g., reagent), kits and methods for high-throughput detection of the TCR sequence at single cell level are disclosed herein. In some embodiments, the compositions, kits and methods are inexpensive and easy to obtain, so effectively reduces costs; the operation process is simple, no special equipment is needed, therefore it can be carried out in ordinary laboratories. The compositions, kits, methods, and systems provided herein allows obtaining TCR and transcriptome information at the same time.

### Detection of Viral Sequences in Host Cells

Methods, compositions, kits and systems are disclosed herein for detecting expressed viral genes and host genes simultaneously at single cell resolution. First, we use probe binding to virus sequence combined with oligo-dT to capture and reverse transcribe expressed viral genes and host mRNA, respectively. The probe and oligo-dT, for example, can contain the same PCR handle sequence, so that cDNA of virus sequence and host mRNA can be amplified at the same time. Optionally, the probe and oligo-dT can be combined with a oligonucleotide sequence that can act as cell barcode to distinguish single cells from each other, so that thousands or more of single cells can be analyzed in parallel. This method can also be used in combination with a microfluidic system where each cell in a sample can be partitioned to individual micro-chambers. Single cells can be lysed in the micro-chambers; mRNA and virus sequences can be captured at the same time.

The methods, compositions, kits and systems disclosed herein can also allow high-throughput detection of the viral sequence at single cell level. Probe binding to virus sequence can be combined with oligo-dT to capture host mRNA and virus nucleotide in a single cell. The probe sequence can be subsequently used to capture said RNA and prime reverse transcription of the RNA to cDNA. The resulting cDNA can be amplified and analyzed. In some embodiments, methods, compositions, kits and systems allows sequencing and quantifying the whole transcriptome of single cells together with the viral RNA from the same single cell.

Hundreds of virus species are known to be able to infect humans, and at least three to four new species emerge every year. Many viruses transmitted in human have mammalian or avian animal origins. Indeed, a substantial proportion of mammalian viruses may be capable of crossing the species barrier into humans, although only around half of these are capable of being transmitted by humans and around half again of transmitting well enough to cause major outbreaks. Recently, the 2019 novel coronavirus (2019-nCoV; or severe acute respiratory syndrome coronavirus 2 (SARS-CoV-2)) has spread rapidly since its recent identification in patients with severe pneumonia in Wuhan, China. As of Feb. 10, 2020, SARS-CoV2 has been reported in 25 countries across 4 continents and >40,000 cases have been confirmed, with an estimated mortality risk of ~2%. Flaviviruses, which include dengue (DENV) and Zika (ZIKV) viruses, infect several hundred million people annually and are associated with severe morbidity and mortality.

Virus infection causes approximately 12% of cancers in the world, Human papilloma virus (HPV), Epstein-Barr virus (EBV), hepatitis B virus (HBV),Kaposi’s sarcoma-associated herpes virus (KSHV),Merkel cell polyomavirus (MCPyV), hepatitis C virus (HCV), Human immunodeficiency virus (HIV) and human T cell lymphotropic virus type 1 (HTLV-1) are associated with multiple forms of malignancies.

High-throughput sequencing (HTS) with next generation sequencing (NGS) has become more common in virus discovery applications. Three main methods based on HTS are currently used for viral RNA sequencing: metatranscriptomics sequencing, target enrichment sequencing and PCR amplicon sequencing. However, these approaches cannot provide accurate information on interaction dynamics between viruses and the host cells.

### Detection of Single Cell Cancer Mutations

Meta-transcriptomics sequencing has been widely used for virus identification and virus-host interactions analysis. Based on the sequence of the virus, it is possible to analyze the characteristics and evolutionary relationship of the virus, so as to know its pathogenic mechanism. For example, high throughput meta-transcriptomic sequencing can be used to obtain complete viral genome sequence of COVID-19. It has been proved COVID-19 was approximately 79% similar to SARS-CoV at the nucleotide level based on sequence alignment. Given these close evolutionary relationships, it has been found that COVID-19 uses the SARS-CoV receptor ACE2 for entry.

However, analyses at the cell population level may average and minimize individual cellular differences, potentially masking rare cells or cell subsets with a significant specific phenotype. This can be found in cancer, where heterogeneity in intra-tumor cells at genetic, epigenetic and phenotypic level can lead to resistance in cancer therapies, as well as in infectious diseases where cell heterogeneity can reveal differential susceptibility to infections or different immunological responses. Furthermore, such bulk sequencing methods do not take into consideration that it is likely that only a small percentage of cells in a host tissue is infected by virus.

The characterization of cellular heterogeneity due to the activation of different host pathways by viral infection and the progression of viral infection is of great interest. Since viruses usurp the cellular machinery at every stage of their life cycle, a therapeutic strategy is to target host factors essential for viral replication. To this end it is important to understand the interaction dynamics between viruses and the infected host cells, to identify pro- and antiviral host factors and to monitor their dynamics in the course of viral infection.

Single-cell transcriptome sequencing is the most popular technology in the field of biology in recent years. Its ultra-high resolution enables accurate analysis of sample information, and has huge application potential in many fields of biology. For example, the heterogeneity of tumors has an important impact on disease development and drug intervention. However, conventional high-throughput sequencing solutions cannot reveal the heterogeneity of tumors. At present, single-cell sequencing has been widely used in tumor microenvironment and immune cell diversity research. In addition, the application fields of single-cell transcriptome sequencing are also expanding, such as the application of early-stage cancer markers, the drug resistance mechanism of tumor targeted therapy, drug target development and expansion of the scope of drug application and so on. Although the single-cell transcriptome technology has been rapidly developed and widely used recently, current technology still cannot fully reveal the integrity and complexity of the transcriptome expression profile, and there is still room for further improvement. Like single-cell targeting panel technology, which aim to obtain more expression information of genes of interest with a limited sequencing depth and further improve the accuracy of single-cell sequencing, such as 10x Genomics-targeted gene expression panel and BD Rhapsody targeting Panel technology. 10x Genomics designs a specific probe panel to achieve the enrichment of target genes by capturing the constructed library. BD Rhapsody uses a multiplex PCR scheme to design a gene specific primer for the gene of interest. After obtaining full-length cDNA, multiplex PCR is performed to capture the target genes. Although these two technologies can improve the detection efficiency of target genes, they still cannot detect the mutation information of target genes far away from the 3′ end of mRNA at the single cell level. In fact, single-cell transcriptome combined with hotspot mutation detection of target genes has a broad application prospect.

Many cancer patients do not have obvious clinical manifestations in the early stages, such as lung cancer. Nearly 60% of lung cancer patients had metastases at the time of initial diagnosis, so they lose the opportunity for early surgical treatment, leading to a poor prognosis. With the development of tumor molecular biology, the application of targeted drugs has significantly improved the prognosis of patients. For example, epidermal growth factor receptor tyrosine kinase inhibitors (EGFR-TKI), such as gefitinib and erlotinib, are gradually being used in patients with advanced non-small cell lung cancer (NSCLC). Due to their low side effects, high safety and good tolerability, the quality of life and overall survival rate of patients with advanced non-small cell lung cancer are improved. A large number of studies around the world have confirmed that EGFR gene mutations in patients with non-small cell lung cancer are a necessary prerequisite for effective targeted therapy of EGFR tyrosine kinase inhibitors (EGFR-TKI). Furthermore, some cancers are often accompanied by a series of gene mutations, such as BRAF, ALK, and NRAS. The occurrence of these mutations will have a significant impact on the therapeutic effect of cancer patients.

Disclosed herein are methods, compositions, kits and systems for detecting target genes of interest and normal transcripts simultaneously at single cell resolution. For example, probe binding to target sequence can be combined with oligo-dT to capture and reverse transcribe target sequence and transcriptome, respectively. The probe and oligo-dT contain the same PCR handle sequence, so that cDNA of target sequence and regular transcripts can be amplified at the same time. Optionally, the probe and oligo-dT can be combined with an oligonucleotide sequence that can act as cell barcode to distinguish single cells from each other, so that thousands or more of single cells can be analyzed in parallel. This method can be used in combination with a microfluidic system where each cell in a sample can be partitioned to individual micro-chambers. For example, single cell can be lysed in the micro-chambers; mRNA and target sequences can be captured at the same time. The methods, compositions, kits and systems can be used, for example, to detect lung cancer druggable mutations in single cells.

Disclosed herein include a method for analyzing targeted regions at single cell level, comprising: (a) capturing the RNA from a single cell with an oligo-dT primer combined with probe sequence that binding to targeted sequence; (b) reverse transcribing the RNA to cDNA with the oligo-dT primer and targeted specific primer; (c) amplifying cDNA; (d) analyzing the amplified cDNA; and (e) enriching the target sequence with specific primers. Provided herein includes a product that includes reagents needed to enable the process for analyzing targeted regions at single cell level.

Single-cell transcriptome sequencing combined with single-cell mutation sequencing technology can simultaneously analyze the cell types and cell mutation information of the transcriptome, which is a powerful tool for studying the relationship between tumor cell development, targeted drugs and gene hotspot mutations. The single-cell transcriptome combined with targeted mutations can accurately identify the cell types that have mutations and provide references for clinical medication. At the same time, it can dynamically monitor changes in the type and frequency of mutations during medication. This technology is realized by coupling magnetic beads with specific capture probes containing cell barcode, UMI, polyT and gene specific primer, and based on the unique single-cell microfluidic system of Singleron, which not only detects ordinary single-cell transcriptomes, the capture probe of the target gene can improve the efficiency of capturing the target gene region. Furthermore, primers for the hotspot region of the target gene are designed. The methods disclosed herein can not only obtain high-quality single-cell transcriptome data, but also information about hotspot mutations of interest at a much lower sequencing depth than the transcriptome according to customer needs. This technology has the following characteristics: (1) High-throughput: it can detect mutations in the region of interest of thousands of cells at the same time; (2) Deep customization: the corresponding capture probe can be designed according to the different needs of customers; (3) Cost-effective: The experimental procedure is highly compatible with the single-cell transcriptome workflow. It only needs to customize the capture magnetic beads and construct the corresponding enrichment library to achieve the capture of the target region.

Disclosed herein include methods and reagents for high-throughput detection of the target region and whole transcriptome simultaneously at single cell level. For example, probe binding to the interested region is combined with oligo-dT to capture whole mRNA in a single cell. The probe sequence is subsequently used to capture said RNA and prime reverse transcription of the RNA to cDNA. The resulting cDNA can be amplified and analyzed. The methods allow sequencing and quantifying the whole transcriptome of single cells together with the target specific RNA from the same single cell. The primers for obtaining more information about the target region with low sequencing depth were designed.

The targeted capture system described herein can be customized and does not rely on polyT capture, which has many advantages, including (1) it can be applied to multiple fields of single cell, such as single cell tumor mutation detection, single cell fusion gene detection, single cell virus detection, single cell lncRNA sequencing, (2) targeted capture at the mRNA level can improve the capture efficiency of target genes, (3) focus on areas of interest to generate smaller and easier-to-manage data sets, (4) reduce the cost of sequencing and the burden of data analysis, (5) faster turnaround time compared to broader methods, and (6) achieved deep sequencing with a high coverage level, suitable for the identification of rare variants.

### Single Cell Analysis and Sequencing

Disclosed herein include methods for single cell analysis. In some embodiments, a method for single cell analysis comprises partitioning a cell and a bead attached with a plurality of barcode oligonucleotides into a partition. Each barcode oligonucleotide of the plurality of barcode oligonucleotides can comprise a cell barcode and a unique molecular identifier (UMI). First barcode oligonucleotides of the plurality of barcode oligonucleotides each can comprise a poly-dT sequence. The poly-dT sequence can be capable of binding to a poly-A region (e.g., a poly-A tail) of a first nucleic acid target (e.g., a first messenger ribonucleic acid (mRNA) target). Second barcode oligonucleotides of the plurality of barcode oligonucleotides each can comprise a poly-dT sequence and a probe sequence. The probe sequence, for example, is not a poly-dT sequence. The probe sequence can include a stretch of thymine (T) bases and additional sequences such that the probe sequence is not a poly-dT sequence. The probe sequence can be capable of binding to a second nucleic acid target (e.g., a second RNA target) at a sequence that is not a poly-A sequence. The method can comprise hybridizing the first barcode oligonucleotides and the second barcode oligonucleotides attached to the bead in the partition with nucleic acid targets (e.g., RNA targets) associated with the cell in the partition. A nucleic acid target can be from the cell. For example, the nucleic acid target be a nucleic acids of the cell, such as an mRNA of the cell. As another example, the nucleic acid target can be a nucleic acids not of the cell, such as an RNA of an virus that has infected the cell. As another example, the nucleic acid target can include an oligonucleotide attached to a protein present in the cell. A nucleic acid target can be in the cell (which can be released from the cell by cell lysis before the nucleic acid target is barcoded). A nucleic acid target can be the surface of the cell (e.g., an oligonucleotide attached to an antibody bound to an antibody on the surface of the cell).

The method can comprise extending the first barcode oligonucleotides and the second barcode oligonucleotides using the nucleic acid targets hybridized to the first barcode oligonucleotides and the second barcode oligonucleotides as templates to generate barcoded nucleic acids. For example, the method can comprise reverse transcribing the RNAs hybridized to the first barcode oligonucleotides and the second barcode oligonucleotides to generate barcoded complementary deoxyribonucleic acids (cDNAs). The method can comprise amplifying the barcoded nucleic acids (e.g., the barcoded cDNAs) to generate amplified barcoded nucleic acids. The method can comprise analyzing the amplified barcoded nucleic acids (e.g., amplified barcoded cDNAs) or products thereof.

In some embodiments, analyzing the amplified barcoded nucleic acids (e.g., amplified barcoded cDNAs) comprises sequencing the amplified barcoded nucleic acids to obtain sequencing information. In some embodiments, analyzing the amplified barcoded nucleic acids comprises determining an expression profile of each of one or more the nucleic acid targets (e.g., RNA targets) using a number of UMIs with different sequences associated with the nucleic target in the sequencing information. For example, analyzing the amplified barcoded nucleic acids can include determining the number of barcoded nucleic acids of each of the nucleic acid targets with UMIs having different sequences in the sequencing information. Analyzing the amplified barcoded nucleic acids (e.g., amplified barcoded cDNAs) can comprise determining an expression profile of the second nucleic acid target (e.g., the second RNA target) using a number of UMIs with different sequences associated with the second nucleic acid target in the sequencing information. For example, analyzing the amplified barcoded nucleic acids can include determining the number of barcoded nucleic acids of the second target with UMIs having different sequences in the sequencing information. The expression profile can comprise an absolute abundance or a relative abundance.

In some embodiments, analyzing the amplified barcoded cDNAs comprises determining a number of amplified barcoded cDNAs of each of one or more the nucleic acid targets (e.g., RNA targets) comprising UMIs with different sequences. Analyzing the amplified barcoded nucleic acid targets can comprise determining a number of amplified barcoded nucleic acid targets of the second RNA target comprising UMIs with different sequences. Analyzing the amplified barcoded cDNAs can comprise determining sequences of the amplified barcoded nucleic acid targets of the second RNA target, or a portion thereof, comprising UMIs with different sequences.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode, (ii) a unique molecular identifier (UMI), and (iiia) a poly-dT sequence and/or (iiib) a probe sequence. The poly-dT sequence can be capable of binding to a poly-A region of a first nucleic acid target. The probe sequence, for example, is not a poly-dT sequence. The probe sequence can include a stretch of thymine (T) bases and additional sequences such that the probe sequence is not a poly-dT sequence. The probe sequence can be capable of binding to a second nucleic acid target. The method can comprise barcoding nucleic acid targets associated with the cell in each partition of the partitions using first barcode oligonucleotides and second barcode oligonucleotides attached to the bead in the partition to generate barcoded nucleic acids. A nucleic acid target can be from the cell. A nucleic acid target can be in the cell. A nucleic acid target can be the surface of the cell. The method can comprise sequencing the barcoded nucleic acids, or products thereof, to obtain sequencing information.

Disclosed herein include methods for single cell sequencing. In some embodiments, a method for single cell sequencing comprises co-partitioning a plurality of cells and a plurality of beads into a plurality of partitions. Partitions of the plurality of partitions each can comprise a single cell of the plurality of cells and a single bead of the plurality of beads. Each of the beads in the partitions of the plurality of partitions can be attached with a plurality of barcode oligonucleotides. Each barcode oligonucleotide of the plurality of barcode oligonucleotides can comprise (i) a cell barcode and (ii) a unique molecular identifier (UMI). The method can comprise barcoding nucleic acid targets associated with the cell in each partition of the partitions to generate barcoded nucleic acids using (a) extension primers and/or a probe sequence and (b) the first barcode oligonucleotides and the second barcode oligonucleotides attached to the bead in the partition. The poly-dT sequence can be capable of binding to a poly-A region of a first nucleic acid target. The probe sequence is not a poly-dT sequence. The probe sequence can be capable of binding to a second nucleic acid target. A nucleic acid target can be from the cell. A nucleic acid target can be in the cell. A nucleic acid target can be the surface of the cell. The first barcode oligonucleotides and the second barcode oligonucleotides attached to the bead in the partition can be used as template switching oligonucleotides for barcoding the nucleic acid targets. The method can comprise sequencing the barcoded nucleic acids, or products thereof, to obtain sequencing information.

In some embodiments, the nucleic acid targets comprise ribonucleic acids (RNAs), messenger RNAs (mRNAs), and/or deoxyribonucleic acid (DNAs). A nucleic acid target can be of the cell, from the cell, in the cell, and/or on the surface of the cell. A nucleic acid target can be from the cell. For example, the nucleic acid target be a nucleic acids of the cell, such as an mRNA of the cell. As another example, the nucleic acid target can be a nucleic acids not of the cell, such as an RNA of an virus that has infected the cell. As another example, the nucleic acid target can include an oligonucleotide attached to a protein present in the cell. A nucleic acid target can be in the cell (which can be released from the cell by cell lysis before the nucleic acid target is barcoded). A nucleic acid target can be the surface of the cell (e.g., an oligonucleotide attached to an antibody bound to an antibody on the surface of the cell). In some embodiments, the method comprises releasing the nucleic acids of (or form or in) the cell prior to barcoding the nucleic acid targets associated with the cell. The method comprises lysing the cell to release the nucleic acids from of (or from or in) the cell.

### Barcode Oligonucleotide Extension (e.g., Reverse Transcription)

In some embodiments, barcoding the nucleic acids associated with the cell comprises hybridizing the first barcode oligonucleotides and the second barcode oligonucleotides attached to the bead in each partition of the partitions with nucleic acid targets associated with the cell in the partition. Barcoding the nucleic acids associated with the cell can comprise extending the first barcode oligonucleotides and the second barcode oligonucleotides attached to the bead and hybridized to the nucleic acid targets using the nucleic acids as templates to generate single-stranded barcoded nucleic acids. For example, the barcoded nucleic acids can be generated by reverse transcription using a reverse transcriptase. For example, the barcoded nucleic acids can be generated by using a DNA polymerase. Barcoding the nucleic acids associated with the cell can comprise generating double-stranded barcoded nucleic acids from the single-stranded barcoded nucleic acids. Extending the single-stranded barcoded nucleic acids comprises further extending the single-stranded barcoded nucleic acids using a template switching oligonucleotide. For example, a reverse transcriptase can be used to generate a cDNA by extending a barcode oligonucleotide hybridized to an RNA. After extending the barcode oligonucleotide to the 5′-end of the RNA, the reverse transcriptase can add one or more nucleotides with cytosine (Cs) bases (e.g., two or three) to the 3′-end of the cDNA. The template switch oligonucleotide (TSO) can include one or more nucleotides with guanine (G) bases (e.g., two or three) on the 3′-end of the TSO. The nucleotides with guanine bases can be ribonucleotides. The guanine bases at the 3′-end of the TSO can hybridize to the cytosine bases at the 3′-end of the cDNA. The reverse transcriptase can further extend the cDNA using the TSO as the template to generate a cDNA with the TSO sequence on its 3′-end. Similarly, a barcoded nucleic acid can include a TSO sequence at its 3′-end.

### Pooling

In some embodiments, the method comprises pooling the beads prior to extending the first barcode oligonucleotides and the second barcode oligonucleotides. The method can comprise pooling the beads prior to generating the double-stranded barcoded nucleic acids. In some embodiments, extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in bulk. Generating the double-stranded barcoded nucleic acids can comprise generating the double-stranded barcoded nucleic acids from the single-stranded barcoded nucleic acids in bulk.

In some embodiments, the method comprises pooling the beads subsequent to extending the first barcode oligonucleotides and the second barcode oligonucleotides to generate the single-stranded barcoded nucleic acids. The method can comprise pooling the beads subsequent to generating the double-stranded barcoded nucleic acids. In some embodiments, extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in the partition. Generating the double-stranded barcoded nucleic acids can comprise generating the double-stranded barcoded nucleic acids from the single-stranded barcoded nucleic acids in the partition.

### Barcoded Nucleic Acid (e.g., cDNA) Amplification

In some embodiments, the method comprises amplifying the barcoded nucleic acid to generate amplified barcoded nucleic acids, such as amplifying barcoded cDNAs. Amplifying the barcoded nucleic acids can comprise amplifying the barcoded nucleic acids using polymerase chain reaction (PCR) to generate the amplified barcoded nucleic acids. For example, the barcode oligonucleotide can include a first polymerase chain reaction (PCR) primer-binding sequence (e.g., a Read 1 sequence) and a TSO sequence. The first PCR primer-binding sequence and the TSO sequence can be used to amplify the barcoded nucleic acid, such as a barcoded cDNA. For example, the barcode oligonucleotide can include a first polymerase chain reaction (PCR) primer-binding sequence (e.g., a Read 1 sequence). A first primer comprising the sequence of first PCR primer-binding sequence and a second primer comprising a random sequence (e.g., a random hexamer) can be used to amplify the barcoded nucleic acid, such as a barcoded cDNA. The second primer can include one or more non-random sequences, such as a second PCR primer-binding sequence (e.g., a Read 2 sequence).

### Enrichment

In some embodiments, the method comprises enriching the one or more second nucleic acid targets using one or more enrichment primers. Enriching the second nucleic acid targets can comprise enriching the second nucleic acid targets using primers specific to the second nucleic acid targets when amplifying the barcoded nucleic acids. For example, a first primer comprising the sequence of first PCR primer-binding sequence and a second primer comprising a sequence specific to a second nucleic acid target (e.g., a partial sequence of the second nucleic acid target, or a reverse complement thereof) can be used to amplify the second barcoded nucleic acid. The second primer can include additional one or more sequences, such as a second PCR primer-binding sequence (e.g., a Read 2 sequence). Enriching the second nucleic acid targets can comprise enriching the second nucleic acid targets using the enrichment primers of a panel. The panel can be a customizable panel.

### Sequencing Library Construction

In some embodiments, the method comprises processing barcoded nucleic acids to generate processed barcoded nucleic acids. For example, the method can include enzymatic fragmentation of the barcoded nucleic acids, end repair of fragmented nucleic acids, A-tailing of fragmented nucleic acids that have been end-repaired, and ligation of a double stranded adaptor with a second PCR primer-binding sequence (e.g., a Read 2 sequence). Sequencing the barcoded nucleic acids can comprise sequencing the processed barcoded nucleic acids.

In some embodiments, processing the amplified barcoded nucleic acids comprises fragmenting the amplified barcoded nucleic acids to generate fragmented barcoded nucleic acids. Fragmenting the amplified barcoded nucleic acids can comprise fragmenting the amplified barcoded nucleic acids enzymatically to generate the fragmented barcoded nucleic acids. Fragmented barcoded nucleic acids can undergo end-repair and A-tailing (to add a few nucleotides with adenosine (A) bases). Processing the amplified barcoded nucleic acids can comprise adding a second polymerase chain reaction (PCR) primer-binding sequence. The second PCR primer-binding sequence can comprise a Read 2 sequence. For example, a double-stranded adaptor comprising the second PCR primer-binding sequence can be ligated to the fragmented barcoded nucleic acids after, for example, end repair and A tailing using a ligase. The adaptor can include a few thymine (T) bases that can hybridize to the few A bases added by A tailing. Processing the amplified barcoded nucleic acids can comprise generating processed barcoded nucleic acids comprising sequencing primer sequences from the fragmented barcoded nucleic acids (e.g., after end repair, A tailing, and ligation of an adaptor comprising the second PCR primer-binding sequence) using PCR. The sequencing primer sequences can comprise a P5 sequence and a P7 sequence. For example, a pair of PCR primers can be sued to add the sequencing primer sequences. A first PCR primer can comprise a P5 sequence and a Read 1 sequence (from 5′-end to 3′-end. A second PCR primer can comprise a P7 sequence and a Read 2 sequence (from 5′-end to 3′-end). A second PCR primer can comprise a P7 sequence, a sample index, and a Read 2 sequence (from 5′-end to 3′-end). The pair of PCR primers can be used to generate processed nucleic acids by PCR. The processed nucleic acids can include a P5 sequence, a Read 1 sequence, a cell barcode, a UMI, a poly-dT sequence, a probe sequence, a sequence of a nucleic acid target or a portion thereof, a Read 2 sequence, a sample index, and/or a P7 sequence (e.g., from 5′-end to 3′-end). In some embodiments, sequencing the barcoded nucleic acids, or products thereof, comprises sequencing products of the barcoded nucleic acids. Products of the barcoded nucleic acids can include the processed nucleic acids.

### Analysis

In some embodiments, the method comprises analyzing the sequencing information. In some embodiments, analyzing the sequencing information comprises determining a profile (e.g., an expression profile) of each of one or more nucleic acid targets of the nucleic acid targets associated with the cell using a number of UMIs with different sequences associated with the nucleic acid target in the sequencing information. Analyzing the sequencing information can comprise determining a profile of the second nucleic acid target using a number of UMIs with different sequences associated with the second nucleic acid target in the sequencing information. A profile can be a single omics profile, such as a transcriptome profile. The profile can be a mutli-omics profile, which can include profiles of a genome, proteome, transcriptome, epigenome, metabolome, and/or microbiome. The profile can include an RNA expression profile. The profile can include a protein expression profile. The expression profile can comprise an absolute abundance or a relative abundance. The expression profile can comprise an RNA expression profile, an mRNA expression profile, and/or a protein expression profile.

Analyzing the sequencing information can comprise determining sequences of the second nucleic acid target, or a portion thereof, associated with UMIs with different sequences. For example, analyzing the sequencing information can include determining presence of one or more mutations (such as an insertion, a deletion, or a substitution) and an abundance (e.g., frequency or occurrence) of each of the mutation. The mutations can be, for example, related to cancer. For example, analyzing the sequencing information can include determining presence of each of one or more variants of a virus and an abundance (e.g., frequency or occurrence) of each variant. The variants can, for example, affect the transmissibility of the virus or affect the severity of the disease caused by the virus. For example, analyzing the sequencing information can include determining the sequences of genes of interest (e.g., TCR alpha and TCR beta) in the cell.

### Partitions

In some embodiments, a partition is a droplet or a microwell. The plurality of partitions can comprise a plurality of microwells of a microwell array. A partition can be sized to fit at most one bead (and one cell), not two beads. A size or dimension (e.g., length, width, depth, radius, or diameter) of a partition can be different in different embodiments. In some embodiments, a size or dimension of one, one or more, or each, of the plurality of partitions is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (µm), 3 µm, 4 µm, 5 µm, 6 µm, 7 µm, 8 µm, 9 µm, 10 µm, 20 µm, 30 µm, 40 µm, 50 µm, 60 µm, 70 µm, 80 µm, 90 µm, 100 µm, 110 µm, 120 µm, 130 µm, 140 µm, 150 µm, 160 µm, 170 µm, 180 µm, 190 µm, 200 µm, 210 µm, 220 µm, 230 µm, 240 µm, 250 µm, 260 µm, 270 µm, 280 µm, 290 µm, 300 µm, 310 µm, 320 µm, 330 µm, 340 µm, 350 µm, 360 µm, 370 µm, 380 µm, 390 µm, 400 µm, 410 µm, 420 µm, 430 µm, 440 µm, 450 µm, 460 µm, 470 µm, 480 µm, 490 µm, 500 µm, or a number or a range between any two of these values. For example, a size or dimension of one, one or more, or each, of the plurality of partitions is about 1 nm to about 100 µm.

The volume of one, one or more, or each, of the plurality of partitions can be different in different embodiments. The volume of one, one or more, or each, of the plurality of partitions can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 µm³, 1000 nm³, 10000 nm³, 100000 µm³, 1000000 nm³, 10000000 nm³, 100000000 µm³, 1000000000 nm³, 2 µm³, 3 µm³, 4 µm³, 5 µm³, 6 µm³, 7 µm³, 8 µm³, 9 µm³, 10 µm³, 20 µm³, 30 µm³, 40 µm³, 50 µm³, 60 µm³, 70 µm³, 80 µm³, 90 µm³, 100 µm³, 200 µm³, 300 µm³, 400 µm³, 500 µm³, 600 µm³, 700 µm³, 800 µm³, 900 µm³, 1000 µm³, 10000 µm³, 100000 µm³, 1000000 µm³, or a number or a range between any two of these values. The volume of one, one or more, or each, of the plurality of partitions can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of partitions is about 1 nm³ to about 1000000 µm³.

The number of partitions can be different in different embodiments. In some embodiments, the number of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of partitions can be at least 1000 partitions.

The percentage of the plurality of partitions comprising a single cell and a single bead can be different in different embodiments. In some embodiments, the percentage of the plurality of partitions comprising a single cell and a single bead is, is about, is at least, is at least about, is at most, or is at most about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 50% of partitions of the plurality of partitions comprise a single cell of the plurality of cells and a single bead of the plurality of beads.

The percentage of the plurality of partitions comprising no cell or two or more cells of the plurality of cells can be different in different embodiments. In some embodiments, the percentage of the plurality of partitions comprising no cell or two or more cells of the plurality of cells is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 10% of partitions of the plurality of partitions can comprise two or more cells of the plurality of cells. As another example, at most 10% of partitions of the plurality of partitions can comprise no cell of the plurality of cells.

The percentage of the plurality of partitions comprising no bead or two or more beads of the plurality of beads can be different in different embodiments. In some embodiments, the percentage of the plurality of partitions comprising no bead or two or more beads of the plurality of beads is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. For example, at most 10% of partitions of the plurality of partitions can comprise two or more beads of the plurality of beads. For example, at most 10% of partitions of the plurality of partitions can comprise no bead of the plurality of beads.

### Beads and Barcode Oligonucleotides

### Beads

In some embodiments, barcode oligonucleotides of the plurality of barcode oligonucleotides are reversibly attached to, covalently attached to, or irreversibly attached to the bead. In some embodiments, the bead is a gel bead. The gel bead can be degradable upon application of a stimulus. The stimulus can comprise a thermal stimulus, a chemical stimulus, a biological stimulus, a photo-stimulus, or a combination thereof. In some embodiments, the bead is a solid bead. The bead can be a magnetic bead.

A bead can be sized such that at most one bead (and one cell), not two beads, can fit one partition. A size or dimension (e.g., length, width, depth, radius, or diameter) of a bead can be different in different embodiments. In some embodiments, a size or dimension of one, or each, bead is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (µm), 3 µm, 4 µm, 5 µm, 6 µm, 7 µm, 8 µm, 9 µm, 10 µm, 20 µm, 30 µm, 40 µm, 50 µm, 60 µm, 70 µm, 80 µm, 90 µm, 100 µm, 110 µm, 120 µm, 130 µm, 140 µm, 150 µm, 160 µm, 170 µm, 180 µm, 190 µm, 200 µm, 210 µm, 220 µm, 230 µm, 240 µm, 250 µm, 260 µm, 270 µm, 280 µm, 290 µm, 300 µm, 310 µm, 320 µm, 330 µm, 340 µm, 350 µm, 360 µm, 370 µm, 380 µm, 390 µm, 400 µm, 410 µm, 420 µm, 430 µm, 440 µm, 450 µm, 460 µm, 470 µm, 480 µm, 490 µm, 500 µm, or a number or a range between any two of these values. For example, a size or dimension of one, or each, bead is about 1 nm to about 100 µm.

The volume of one, or each, bead can be different in different embodiments. The volume of one, or each, bead can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 µm³, 1000 nm³, 10000 nm³, 100000 µm³, 1000000 nm³, 10000000 nm³, 100000000 µm³, 1000000000 nm³, 2 µm³, 3 µm³, 4 µm³, 5 µm³, 6 µm³, 7 µm³, 8 µm³, 9 µm³, 10 µm³, 20 µm³, 30 µm³, 40 µm³, 50 µm³, 60 µm³, 70 µm³, 80 µm³, 90 µm³, 100 µm³, 200 µm³, 300 µm³, 400 µm³, 500 µm³, 600 µm³, 700 µm³, 800 µm³, 900 µm³, 1000 µm³, 10000 µm³, 100000 µm³, 1000000 µm³, or a number or a range between any two of these values. The volume of one, or each, bead can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, or each, bead is about 1 nm³ to about 1000000 µm³.

The number of beads can be different in different embodiments. In some embodiments, the number of beads is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of beads can be at least 1000 beads.

### Poly-dT Sequence and Probe Sequence

The number of the barcode oligonucleotides (or the number of first barcode oligonucleotides each comprising a poly-dT sequence, the number of second barcode oligonucleotides each comprising a probe sequence, or the number of second barcode oligonucleotides comprising a particular probe sequence) attached to a bead can be different in different embodiments. In some embodiments, the number of barcode oligonucleotides (or the number of first barcode oligonucleotides each comprising a poly-dT sequence, the number of second barcode oligonucleotides each comprising a probe sequence, or the number of second barcode oligonucleotides comprising a particular probe sequence) attached to a bead is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

The ratio of (i) first barcode oligonucleotides each comprising a poly-dT sequence and (ii) second barcode oligonucleotides each comprising a probe sequence (or the ratio of (i) first barcode oligonucleotides each comprising a poly-dT sequence and (ii) second barcode oligonucleotides each comprising a particular probe sequence, or the ratio of (i) second barcode oligonucleotides each comprising a first probe sequence and (ii) second barcode oligonucleotides each comprising a second probe sequence) can be different in different embodiments. In some embodiments, the ratio is, is about, is at least, is at least about, is at most, is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values.

The length of a poly-dT sequence can be different in different embodiments. In some embodiments, a poly-dT sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a poly-dT sequence is at least 10 nucleotides in length.

The length of a probe sequence can be different in different embodiments. In some embodiments, a probe sequence is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a probe sequence is at least 10 nucleotides in length.

In some embodiments, first barcode oligonucleotides of the plurality of barcode oligonucleotides each comprises a poly-dT sequence. The poly-dT sequence can be capable of binding to a poly-A region (e.g., a poly-A tail) of a first nucleic acid target. In some embodiments, the poly-dT sequences of the first barcode oligonucleotides of the plurality of barcode oligonucleotides attached to a bead (or each bead or all beads) are identical. The percentage of the first barcode oligonucleotides of the plurality of barcode oligonucleotides attached to a bead (or each bead or all beads) with an identical poly-dT sequence can be different in different embodiments. In some embodiments, the percentage of the first barcode oligonucleotides of the plurality of barcode oligonucleotides attached to a bead (or each bead or all beads) with an identical poly-dT sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values.

In some embodiments, second barcode oligonucleotides of the plurality of barcode oligonucleotides each comprises a probe sequence. The probe sequence, for example, is not a poly-dT sequence (though a probe sequence can comprise a stretch of Ts). The probe sequence can be capable of binding to a second nucleic acid target. The number of different probe sequences of the barcode oligonucleotides attached to a bead (or each bead or all beads) can be different in different embodiments. In some embodiments, the number of different probe sequences of the barcode oligonucleotides attached to a bead (or each bead or all beads) is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values.

The number of different nucleic acid targets (e.g., mRNAs of different genes or mRNAs of different sequences) the barcode oligonucleotides attached to a bead (or each bead) are capable of binding can be different in different embodiments. In some embodiments, the number of different nucleic acid targets the barcode oligonucleotides attached to a bead (or each bead) are capable of binding is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. One barcode oligonucleotide attached to a bead (or each) can bind to a molecule (or a copy) of a nucleic acid target. Barcode oligonucleotides attached to a bead (or each) can bind to molecules (or copies) of a nucleic acid target.

In some embodiments, second barcode oligonucleotides of the plurality of barcode oligonucleotides each comprises a poly-dT sequence and a probe sequence. The probe sequence, for example, is not a poly-dT sequence. The probe sequence can be capable of binding to a second nucleic acid target. In some embodiments, second barcode oligonucleotides of the plurality of barcode oligonucleotides comprise probe sequences that are not poly-dT sequences. The probe sequences can be capable of binding to an identical second nucleic acid target. In some embodiments, second barcode oligonucleotides of the plurality of barcode oligonucleotides comprise probe sequences that are not poly-dT sequences. The probe sequences can be capable of binding to different second nucleic acid targets.

In some embodiments, the probe sequences of barcode oligonucleotides of the plurality of barcode oligonucleotides comprise a degenerate sequence. The length of a degenerate sequence can be different in different embodiments. In some embodiments, the length of the degenerate sequence is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. For example, a length of the degenerate sequence can be at least 3. The degenerate sequence can span a mutation. For example, the degenerate sequence is three nucleotides in length, and the second position of the degenerate sequence is the position of a single nucleotide variation. The degenerate sequence can correspond a mutation. For example, the degenerate sequence is one nucleotide in length, and the position of the degenerate sequence corresponds to the position of a single nucleotide variation. The length of the degenerate sequence and the length of the mutation can be identical. The length of the degenerate sequence and the length of the mutation can be different. The length of the degenerate sequence can be longer the length of the mutation.

In some embodiments, a probe sequence of a barcode oligonucleotide of the plurality of barcode oligonucleotides spans a region of interest. In some embodiments, a probe sequence of a barcode oligonucleotide of the plurality of barcode oligonucleotides corresponds a region of interest. In some embodiments, the probe sequence is adjacent (upstream or downstream) a region of interest.

The region of interest can comprise a variable region of a T-cell receptor (TCR). The TCR can be TCR alpha or TCR beta. In some embodiments, the region of interest comprises a mutation. In some embodiments, the mutation comprises an insertion, a deletion, or a substitution. The substitution can comprise a single-nucleotide variant (SNV) or a single-nucleotide polymorphism (SNP). The mutation can be related to a disease, such as a cancer. A bead attached thereto second oligonucleotide barcodes having probe sequences for binding to disease-related (e.g., cancer-related) genes is referred to herein as a druggable bead. The mutations of the genes are referred to herein as druggable mutations.

### Cell Barcode

The number (or percentage) of barcode oligonucleotides attached to a bead with cell barcodes having an identical sequence can be different in different embodiments. In some embodiments, the number of barcode oligonucleotides attached to a bead with cell barcodes having an identical sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode oligonucleotides attached to a bead with cell barcodes having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, the cell barcodes of two (or more) barcode oligonucleotides attached to a bead comprise an identical sequence.

A cell barcode can be unique (or substantially unique) to a bead. The number (or percentage) of beads with cell barcodes having unique sequences can be different in different embodiments. In some embodiments, the cell barcodes of, of about, of at least, of at least about, of at most, or of at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values, beads can comprise different sequences. In some embodiments, the cell barcodes of, of about, of at least, of at least about, of at most, or of at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values, of the beads can comprise different sequences. For example, the cell barcodes of two barcode oligonucleotides attached to two beads can comprise different sequences.

The length of a cell barcode of a bead (or each cell barcode of a bead or all cell barcodes of all beads) can be different in different embodiments. In some embodiments, a cell barcode of a bead (or each cell barcode of a bead or all cell barcodes of all beads) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a cell barcode can be at least 6 nucleotides in length.

The number of unique cell barcode sequences can be different in different embodiments. In some embodiments, the number of unique cell barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value.

### UMI

The number (or percentage) of UMIs of barcode oligonucleotides attached to a bead with different sequences can be different in different embodiments. In some embodiments, the number of UMIs of barcode oligonucleotides attached to a bead with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value. In some embodiments, the percentage of UMIs of barcode oligonucleotides attached to a bead with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, the UMIs of two barcode oligonucleotides attached to a bead of the beads can comprise different sequences.

The number of barcode oligonucleotides attached to a bead with UMIs having a particular sequence (or an identical sequence) can be different in different embodiments. In some embodiments, the number of barcode oligonucleotides attached to a bead with UMIs having a particular sequence (or an identical sequence) is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the UMIs of two barcode oligonucleotides attached to a bead can comprise a particular sequence (or an identical sequence).

Barcode oligonucleotides attached to different beads can have UMIs with a particular sequence (or an identical sequence). In some embodiments, the number of beads attached thereto barcode oligonucleotides having UMIs with a particular sequence (or an identical sequence) is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value. For example, the UMIs of two barcode oligonucleotides attached to two beads of the beads can comprise an identical sequence.

The length of a UMI of a bead (or each UMI of a bead or all UMIs of all beads) can be different in different embodiments. In some embodiments, a UMI of a bead (or each UMI of a bead or all UMIs of all beads) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a UMI can be at least 6 nucleotides in length.

The number of unique UMI sequences can be different in different embodiments. In some embodiments, the number of unique UMI sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value.

### PCR Primer-Binding Sequence

In some embodiments, each barcode oligonucleotide of the plurality of barcode oligonucleotides comprises a first polymerase chain reaction (PCR) primer-binding sequence. The first PCR primer-binding sequence can comprise a Read 1 sequence.

### Targets

The number of different second nucleic acid targets (e.g., mRNAs of different genes or mRNAs of different sequences) the second barcode oligonucleotides with probe sequences are capable of binding can be different in different embodiments. In some embodiments, the number of different second nucleic acid targets the second barcode oligonucleotides with probe sequences are capable of binding is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. For example, second barcode oligonucleotides attached to a bead can have probe sequences capable of binding to 10 target nucleic acids (e.g., multiple molecules or copies of each target nucleic acid). One second barcode oligonucleotide attached to a bead (or each) can bind to a molecule (or a copy) of a second nucleic acid target. Second barcode oligonucleotides attached to a bead (or each) can bind to molecules (or copies) of a second nucleic acid target.

In some embodiments, the second nucleic acid target comprises no poly-A tail and/or no poly-A region. In some embodiments, the second nucleic acid target comprises a poly-A region, The poly-A region can be a poly-A tail.

In some embodiments, the second nucleic acid target comprises a T-cell receptor (TCR), or an RNA (e.g., mRNA) product thereof. The probe sequence can be capable of binding to a constant region, or a portion thereof, of the TCR. The TCR can be TCR alpha or TCR beta. The method can thus determine a profile (e.g., RNA expression profile) of a TCR and sequences of the variable region of the TCR.

In some embodiments, the cell is a cancer cell. The second nucleic acid target is a cancer gene (or a disease-related gene), or an RNA (e.g., mRNA) product thereof. The method can thus determine a profile (e.g., RNA expression profile) of a cancer gene (or a disease-related gene), mutations of the gene, and abundances of the mutations.

In some embodiments, a cell is infected with a virus. The second nucleic acid target can be a gene of the virus, or a nucleic acid product (e.g., RNA) thereof. The virus can be an RNA virus. The second nucleic acid target can comprise an RNA of the gene of the virus. The method can thus determine a profile (e.g., an RNA expression profile) of the cell and a nucleic acid profile (e.g., RNA expression profile) of the virus.

In some embodiments, an abundance of molecules of the second nucleic acid target hybridized to (or barcoded using) the second barcode oligonucleotides is higher than an abundance of molecules of the second nucleic acid target hybridized to (or barcoded using) the first barcode oligonucleotides. The method can thus enrich the second nucleic acid target.

An abundance can be a number or a frequency of occurrences. In some embodiments, the abundance of the molecules of the second nucleic acid target comprises a number of occurrences of the molecules of the second nucleic acid target. In some embodiments, the abundance of the molecules of the second nucleic acid target can comprise a number of occurrences of the molecules of the second nucleic acid target relative to a number of the first barcode oligonucleotides or a number of the second barcode oligonucleotides.

### Composition & Kit

Disclosed herein include compositions for single cell sequencing or single cell analysis. In some embodiments, a composition for single cell sequencing or single cell analysis comprises a plurality of beads of the present disclosure. The cell barcodes of the plurality of barcode oligonucleotides attached to each of the plurality of beads can be identical. The cell barcodes of barcodes oligonucleotide attached to different beads of the plurality of beads can be different. The number of beads can be different in different embodiments. In some embodiments, the number of beads is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number beads can be at least 100 beads.

Disclosed herein include kits for single cell sequencing or single cell analysis. In some embodiments, a kit for single cell sequencing or single cell analysis comprises a composition comprising a plurality of beads of the present disclosure. The kit can comprise instructions of using the composition for single cell sequencing or single cell analysis.

Disclosed herein includes methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode, a unique molecular identifier (UMI), and a poly-dT sequence. The method can comprise adding, to 3′-end of each of barcode oligonucleotides of the plurality of barcode oligonucleotides, a probe sequence that is a not poly-dT sequence and is capable of binding to a nucleic acid target.

In some embodiments, adding the probe sequence comprises adding the probe sequence to the 3′-end of each of the barcode oligonucleotides of the plurality of barcode oligonucleotides chemically. In some embodiments, adding the probe sequence comprises adding the probe sequence to the 3′-end of each of the barcode oligonucleotides of the plurality of barcode oligonucleotides using an enzyme. In some embodiments, the enzyme is a ligase. Adding the probe sequence can comprise ligating a probe oligonucleotide comprising the probe sequence to the 3′-end of each of the barcode oligonucleotides of the plurality of barcode oligonucleotides using the ligase. In some embodiments, the enzyme is a DNA polymerase. Adding the probe sequence can comprise synthesizing the probe sequence at the 3′-end of each of the barcode oligonucleotides of the plurality of barcode oligonucleotides using the DNA polymerase.

Disclosed herein include methods of generating beads comprising barcode oligonucleotides. In some embodiments, a method of generating beads comprising barcode oligonucleotides comprises providing a plurality of beads each attached to a plurality of oligonucleotide barcodes. Each barcode oligonucleotide of the plurality of barcode oligonucleotides can comprise a cell barcode and a unique molecular identifier (UMI). The method can comprise adding to 3′-end of each of barcode oligonucleotides of the plurality of barcode oligonucleotides (i) a poly-dT sequence and/or (ii) a probe sequence that is a non-poly-dT sequence and is capable of binding to a nucleic acid target.

### Cancers and Cancer Genes

The methods, compositions and kits disclosed herein can be used for determining the profile of a cancer gene (e.g., an expression profile of the gene and/or one or more mutations of the gene). A cancer gene can be ABL1, ABL2, ACVR1B, ACVR2A, ADARB2, ADGRA2, ADGRG4, AFDN, AKT1, AKT1S1, AKT2, AKT3, ALB, ALK, ALOX12B, ALOX15B, ALOX5, AMER1, APC, APEX1, AR, ARAF, ARFRP1, ARHGAP35, ARID1A, ARID1B, ARID2, ASXL1, ATM, ATR, ATRX, AURKA, AURKB, AXIN1, AXIN2, AXL, B2M, BAP1, BARD1, BCL2, BCL2L1, BCL2L2, BCL6, BCOR, BCORL1, BCR, BIRC5, BLM, BRAF, BRCA1, BRCA2, BRD2, BRD3, BRD4, BRIP1, BTG1, BTG2, BTK, BUB1B, CARD11, CASP8, CBFB, CBL, CBLB, CCND1, CCND2, CCND3, CCNE1, CD274, CD79A, CD798, CDC7, CDC73, CDH1, COK12, CDK4, CDK6, CDK6, CDKN1A, CDKN1B, CDKN2A, COKN2B, CDKN2C, CEBPA, CEP295, CHEK1, CHEK2, CIC, CNOT3, CREBBP, CRKL, CRTC1, CSF1R, CTCF, CTLA4, CTNNA1, CTNNB1, CUL3, CUX1, CYLD, DAXX, DDIT3, DDR1, DDR2, DEPDC5, DEPTOR, DICER1, DLL4, DNMT3A, DOT1L, DYRK2, E2F3, ECT2L, EGFR, EIF1AX, EIF4A1, EIF4A2, EIF4A3, EIF4B, EIF4E, EIF4E2, ELF3, EML4, EMSY, EP300, EPCAM, EPHA3, EPHA5, EPHA7, EPH8 1, ERBB2, ERBB3, ERBB4, ERCC1, ERCC2, ERCC3, ERCC4, ERCC5, ERCC6, ERCC8, ERG, ERRFI1, ESR1, ETV1, ETV4, ETV5, ETV6, EWSR1, EXO1, EZH2, FAAP100, FAAP20, FAAP24, FAM175A, FAM46C, FANCA, FANCB, FANCC, FANCD2, FANCE, FANCF, FANCG, FANCI, FANCL, FANCM, FAS, FAT1, FBXW7, FEN1, FGF10, FGF14, FGF19, FGF23, FGF3, FGF4, FGF6, FGFR1, FGFR2, FGFR3, FGFR4, FH, FLCN, FLT1, FLT3, FLT4, FOXA1, FOXL2, FOXO1, FOXP1, FRS2, FUBP1, FZD1, FZD10, FZD2, FZD3, FZD4, FZD5, FZD6, FZD7, FZD8, FZD9, GAS6, GATA1, GATA2, GATA3, GATA6, GEN1, GID4, GNA11, GNA13, GNAQ, GNAS, GRIN2A, GSK3B, H3F3A, HDAC2, HELQ, HES1, HEY1, HEYL, HGF, HIST3H3, HNF1A, HRAS, HSP90AA1, IDH1, IDH2, IDO1, IFNG, IFNGR1, IFNGR2, IGF1, IGF1R, IGF2, IGF2R, IKBKE, IKZF1, IL2RG, IL7R, INHBA, INPP4B, IRF1, IRF4, IRS2, JAK1, JAK2, JAK3, JUN, KAT6A, KDM4A, KDM5A, KDM5B, KDM5C, KDM6A, KDR, KEAP1, KIT, KLHL6, KMT2A, KMT20, KNSTRN, KRAS, LGR4, LGR5, LGR6, LIG1, LIG4, LMO1, LRP1B, LRP2, LRP5, LRP6, MAD2L2, MAP2K1, MAP2K2, MAP2K4, MAP3K1, MAP4K3, MAPK1, MAPK3, MAPKAP1, MAX, MCL1, MDC1, MDM2, MDM4, MED12, MEF28, MEN1, MERTK, MET, MITF, MLH1, MLH3, MLST8, MPL, MRAS, MRE11, MSH2, MSH3, MSH6, MTOR, MUTYH, MYB, MYC, MYCL, MYCN, MYD88, NBN, NF1, NF2, NFE2L2, NFKBIA, NHEJ1, NKX2-1, NOTCH1, NOTCH2, NOTCH3, NOTCH4, NPM1, NPRL2, NPRL3, NRAS, NSD1, NTRK1, NTRK2, NTRK3, NUMB, NUP93, NUTM1, PAK3, PALB2, PARG, PARP1, PARP2, PAX5, PBRM1, PCDH15, PDCD1, PDCD1LG2, PDGFRA, PDGFRB, PDK1, PHF6, PIAS4, PIK3C28, PIK3CA, PIK3CB, PIK3CD, PIK3CG, PIK3R1, PIK3R2, PIK3R3, PIM1, PIN1, PKM, PLEKHS1, PMS1, PMS2, POLD1, POLE, POLH, POLQ, POU2F2, PPARG, PPM1D, PPP2CA, PPP2R1A, PPP2R2A, PPP3CA, PPP6C, PRDM1, PREX1, PREX2, PRKAR1A, PRKCI, PRKDC, PTCH1, PTEN, PTPN11, PTPRD, RAC1, RAD18, RAD21, RAD50, RAD51, RAD51B, RAD51C, RAD51D, RAD52, RAD54L, RAF1, RARA, RASA1, RB1, RBM10, RET, REV3L, RGS1, RHEB, RHOA, RHOB, RICTOR, RIT1, RNF43, ROBO1, ROBO2, ROS1, RPA1, RPS27A, RPS6KA3, RPS6KB1, RPTOR, RRAGC, RSPO1, RSPO4, RUNX1, RUNX1T1, SDHB, SDHC, SDHD, SESN2, SETD2, SF3B1, SHFM1, SLC34A2, SLFN11, SLIT2, SMAD2, SMAD3, SMAD4, SMARCA2, SMARCA4, SMARCB1, SMO, SOCS1, SOCS3, SOS1, SOX10, SOX2, SOX9, SPEN, SPOP, SRC, SRSF2, SRY, STAG2, STAT3, STAT4, STK11, STK19, SUFU, SYK, TBC1D7, TBX3, TEK, TERT, TET2, TGFBR2, TMPRSS2, TNFAIP3, TNFRSF14, TNFRSF1A, TNK2, TOP1, TOPAZ1, TP53, TP53BP1, TP63, TP73, TRAF3, TSC1, TSC2, TSHR, TSHZ2, TYRO3, U2AF1, UBE2T, USP9X, VEGFA, VHL, WEE1, WISP3, WRN, WT1, XBP1, XPA, XPC, XP01, XRCC1, XRCC2, XRCC3, XRCC4, XRCC5, XRCC6, YAP1, ZNF217, ZNF703, ZNRF3, or ZRSR2.

The mutation can be related (linked to or cause) to a disease, such as cancer. The cancer can be melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin’s lymphoma (“HL”), Non-Hodgkin’s lymphoma (“NHL”), Follicular lymphoma (“FL”), acute myeloid leukemia (“AML”), and Multiple myeloma (“MM”).

The cancer can be, for example, ovarian cancer, breast cancer, prostate cancer, colorectal cancer, pancreatic cancer, or a combination thereof. In some embodiments, the cancer is ovarian cancer. In some embodiments, the cancer is breast cancer. In some embodiments, the cancer is prostate cancer. The cancer can be a BRCA1 mutant cancer, a BRCA2 mutant cancer, or both. In some embodiments, the cancer is a BRCA2-mutant prostate cancer. In some embodiments, the cancer is a BRCA1-mutatnt ovarian cancer.

### EXAMPLES

Some aspects of the embodiments discussed above are disclosed in further detail in the following example, which are not in any way intended to limit the scope of the present disclosure.

### Example 1 Detection of TCR Sequences

To overcome the drawbacks of the current single cell TCR analysis methods, probe binding to TCR sequence were combined with oligo-dT to capture mRNA while improving the capture efficiency of TCR sequences. The probes and polyT oligo contain the same PCR handle sequence, which can act as priming site for RT reactions and TCR Target enrichment reactions.

For example, probes binding to TCR sequence can be added to the 3′ end of oligo-dT, which allows capturing and reverse transcribing both mRNA and TCR sequence captured by the probes. The resulting cDNA can be used as template to enrich TCR sequence by multiplex PCR. With unique cell barcodes in conjunction with the oligo-dT sequence, cDNA molecules from the same single cell can be labeled and a group of single cells can be processed in parallel. Synergy can be achieved by pairing TCR sequences, which can reveal information about T-cell ancestry and antigen specificity, with information about expression of genes characteristic of particular T-cell functions. Integrating these two types of information enables comprehensive profiling T cells.

In this example, GEXSCOPE Single Cell RNAseq Library Construction kit (Singleron Biotechnologies) was used to show the technical feasibility and the utility of the methods, kits, compositions, and systems in high-throughput single cell ncRNA sequencing. The experiment was conducted according to manufacturer’s instructions with modifications described below.

Cell barcoding magnetic bead synthesis: cell barcoding magnetic beads were synthesized. The primers on all the beads comprise a common sequence used for PCR amplification, a bead-specific cell barcode, a unique 8 molecular identifier (UMI), a oligo-dT sequence for capturing polyadenylated mRNAs and probe sequence annealing to TCR constant Region for capturing TCR mRNA.

The four sequences of the TCR constant region are shown below:
Human T Cell R1-1: TGAAGGCGTTTGCACATGCA (SEQ ID NO: 1)
Human T Cell R1-2: TCAGGCAGTATCTGGAGTCATTGAG (SEQ ID NO: 2)
Human T Cell R2-1: AGTCTCTCAGCTGGTACACG (SEQ ID NO: 3)
Human T Cell R2-2: TCTGATGGCTCAAACACAGC (SEQ ID NO: 4)

The complementary sequences are:
PolyA R1-1: CAAACGCCTTCAAAAAAAAAAAAA (SEQ ID NO: 5)
PolyA R1-2: GATACTGCCTGAAAAAAAAAAAAA (SEQ ID NO: 6)
PolyA R2-1 AGCTGAGAGACTAAAAAAAAAAAA (SEQ ID NO: 7)
PolyA R2-2: TGAGCCATCAGAAAAAAAAAAAAA (SEQ ID NO: 8)

Single cell suspension of PBMC was loaded onto the microchip to partition single cells into individual wells on the chip. Cell barcoding magnetic beads were then loaded to the microchip and washed. Only one bead could fall into each well on the microchip based on the diameters of the beads and well (about 25 um and 40 um, respectively).

100 ul cell lysis buffer was loaded into the chip and let incubate at room temperature for 20 minutes to lyse cells and capture RNAs. After 20 minutes, the magnetic beads, together with captured RNAs, were taken out of the microchip and subject to RT, template switching, cDNA amplification, and a part of cDNA was used to construct Gene expression library using reagents from the GEXSCOPE kit and following manufacturer’s instructions.

The rest of cDNA were used to enrich TCR sequence as described below:

1. Frist-round of enrichment: Take 10 ng cDNA as the template for the first round of TCR enrichment by multiplex nested PCR using QIAGEN Multiplex PCR kit.

Primer design: TCR V region primer (TRV Reaction1) combined with the universal sequence (Target 1F). TCR V region primer (TRV Reaction1) including 38 TRA V regions and 36 TRB V region primers, total 74 primers.

Configure the PCR mix as shown in Table 1 and mix thoroughly:

TABLE 1

PCR mix

| Component | volume (ul) |
|---|---|
| 2x QIA master mix | 25ul |
| TRV Reaction1 primer (50 um) | 4.44 ul |
| Target 1F primer (10 um) | 1.5 ul |
| Dnase/Rnase-Free Water | 19.06-x |
| Template cDNA | x |
| Total | 50 ul |

The final concentration of each TCR V-region primer was 0.06 µM, Target 1F primer was 0.3 µM.

Perform the reaction on the PCR instrument under the conditions as shown in Table 2.

TABLE 2

PCR conditions

| | Temperature | Time |
|---|---|---|
| 1 | 95° C. | 15 min |
| 2 | 94° C. | 30 s |
| 3 | 62° C. | 90 s |
| 4 | 72° C. | 90 s |
| 5 | GOTO Step 2, 9X | |
| 6 | 72° C. | 10 min |
| 7 | 4° C. | hold |

(4) Product purification: 0.8x purification of the obtained PCR product (purification method is the same as above)

2. Second-round of enrichment: a 10-µl aliquot of the first reaction was used as a template for second 50-µl PCR using QIAGEN Multiplex PCR kit;

Primer design: TCR V region primer (TRV Reaction2) combined with the universal sequence (Target 2F), TCR V region primer (TRV Reaction2) includes 36 TRA V region primers, 36 TRB V region primers, total 72 primers.

Configure the PCR mix as shown in Table 3 and mix thoroughly:

TABLE 3

PCR mix

| Component | Volume (ul) |
|---|---|
| 2x QIA master mix | 25 ul |
| TRV Reaction1 primer (50 uM) | 0.6 ul |
| Target 2F primer (10 uM) | 1.5 ul |
| Dnase/Rnase-Free Water | 12.9 ul |
| aliquots of the first-round PCR products | 10 ul |
| Total | 50 ul |

Note: V primers was 0.6 µM, Target 2F primer was 0.3 µM.

Perform the reaction on the PCR instrument under the conditions shown in Table 4.

TABLE 4

PCR conditions

| | Temperature | Time |
|---|---|---|
| 1 | 95° C. | 15 min |
| 2 | 94° C. | 30 s |
| 3 | 62° C. | 90 s |
| 4 | 72° C. | 90 s |
| 5 | GOTO Step 2, 9X | |
| 6 | 72° C. | 10 min |
| 7 | 4° C. | hold |

Product purification: 0.8x purification of the obtained PCR product, for which purification method is the same as described above.

3. Amplification and library construction: Take 20 ng of the second- round enrichment products and use KAPA HiFi PCR kit for amplification and library construction by multiplex PCR (1) Configure the PCR mix as shown in Table 5 and mix thoroughly:

TABLE 5

PCR mix

| Component | Volume (ul) |
|---|---|
| 5X HiFi with Mgcl2 buffer | 10 ul |
| 10 mM dNTP Mix | 1.5 ul |
| HiFi HotStart | 1 ul |
| Target 1F (10 uM) | 1.5 ul |
| PCR-N7XX primer | 1.5 ul |
| Dnase/Rnase-Free Water | 34.5-x ul |
| aliquots of the second-round PCR | x |
| Total | 50 |

Perform the reaction on the PCR instrument under the conditions shown in Table 6.

TABLE 6

PCR conditions

| | Temperature | Time |
|---|---|---|
| 1 | 95° C. | 3 min |
| 2 | 98° C. | 20 s |
| 3 | 64° C. | 30 s |
| 4 | 72° C. | 1 min |
| 5 | GOTO Step 2, 5X | |
| 6 | 72° C. | 5 min |
| 7 | 4° C. | hold |

Product purification: The obtained PCR product was purified 0.8x (the purification method is the same as above), add 20 µl of Nuclease-Free water to elute DNA.

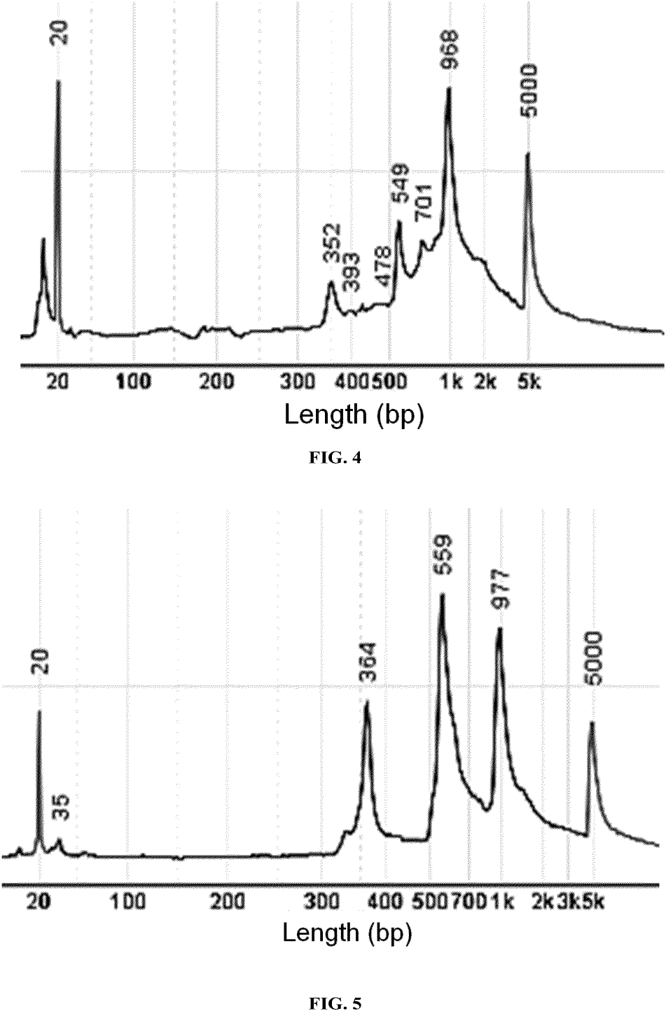

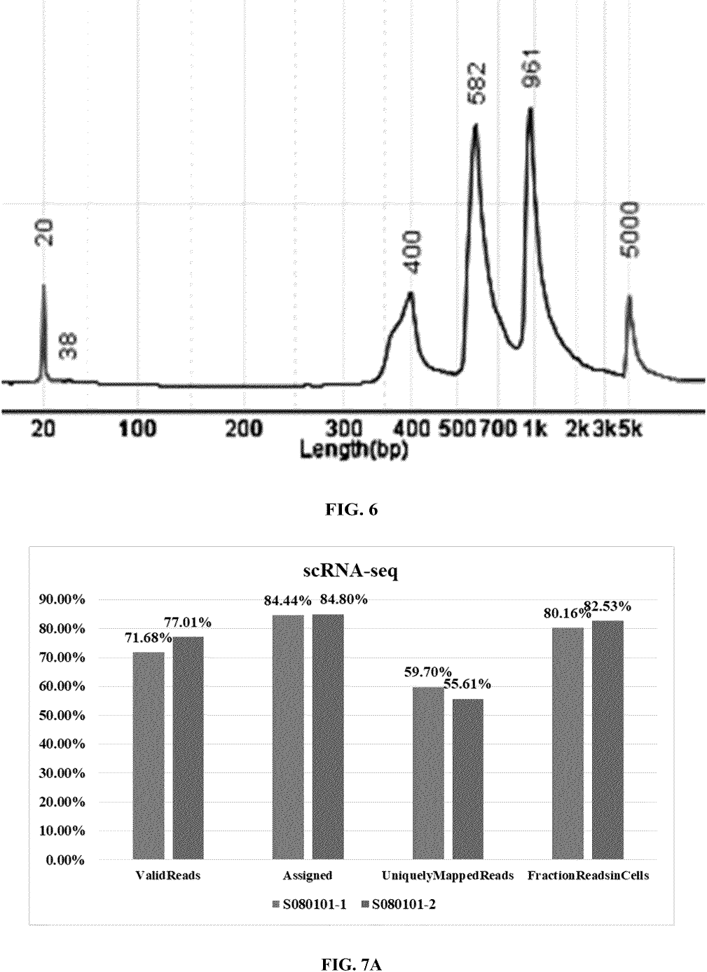

The resulting single cell RNAseq library was sequenced on Illumina NovaSeq with PE150 mode and analyzed with scopeTools bioinformatics workflow (Singleron Biotechnologies).
(1) Amplified cDNA map. FIG. 3 shows the amplified cDNA map.
(2) TCR Target Enrichment 1 map. FIG. 4 shows the TCR target enrichment 1 map.
(3) TCR Target Enrichment 2 map. FIG. 5 shows the TCR target enrichment 2 map.
(4) TCR libray map. FIG. 6 shows the TCR libray map.
(5) TCR Mapping: results are shown in Table 7.

TABLE 7

TCR mapping

| Item | Count | Total Count | Percent |
|---|---|---|---|
| TCR Mapped_read | 5563858 | 5630609 | 98.81% |
| TRA chains: | 2442459 | 5563858 | 43.90% |
| TRB chains: | 2712057 | 5563858 | 48.74% |
| cell_with_matched_barcode | 1990 | — | — |
| matched_cell_with_TRA_and_TRB | 1244 | 1990 | 62.51 |

Top 10 Clonetype Frequencies (Table 8)

TABLE 8

Clonetype Frequencies

| TOP 10 Clonetype | SEQ ID NO | | SEQ ID NO | | |
|---|---|---|---|---|---|
| aSeqCDR3_TRA | | aaSeqCDR3_TRB | | barcode | percent |
| CAFRDYGGSQGNLIF | 1 | CASSLAGAGNEQFF | 2 | 86 | 4.32 |
| CALIIGPGGGTSYGKLTF | 3 | CASSLNRVNEQYF | 4 | 62 | 3.12 |
| NA | | CASSLNRVNEQYF | 12 | 43 | 2.16 |
| CLVGETYNTDKLIF | 9 | CASSIGIGREQYF | 5 | 30 | 1.51 |
| CAYAVGNTGNQFYF | 6 | CAS SYMAGGLNEQFF | 7 | 23 | 1.16 |
| NA | | CSARQTSLYEQYF | 8 | 21 | 1.06 |
| CAFRDYGGSQGNLIF | 9 | NA | | 20 | 1.01 |
| CAGGSEKLVF | 10 | CASSLYQRGVEETQYF | 11 | 18 | 0.9 |
| CAAKTGGGNKLTF | 12 | CASSIFGEQYF | 13 | 15 | 0.75 |
| CAGHNTGKLIF | 14 | CASSLRLDEQYF | 15 | 15 | 0.75 |

The resulting data shows that the mapping rate of TCR can reach more than 90%, and the detection rate of TRA and TRB paired cells also reaches 62%. The number of T cells annotated in the transcriptome data is consistent with the number of T cells detected in the TCR enrichment library.

### Example 2

### Detection of TCR Sequences

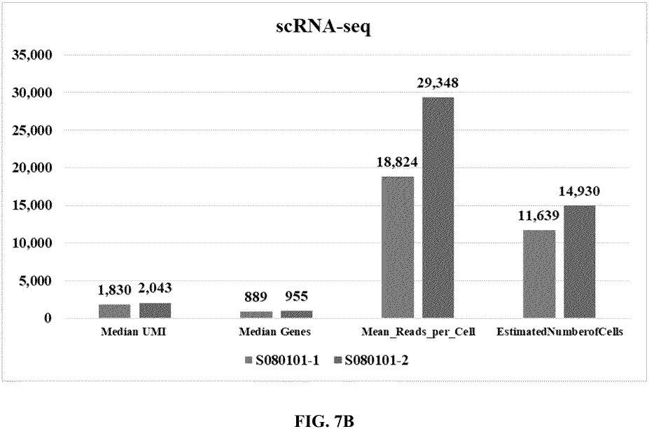

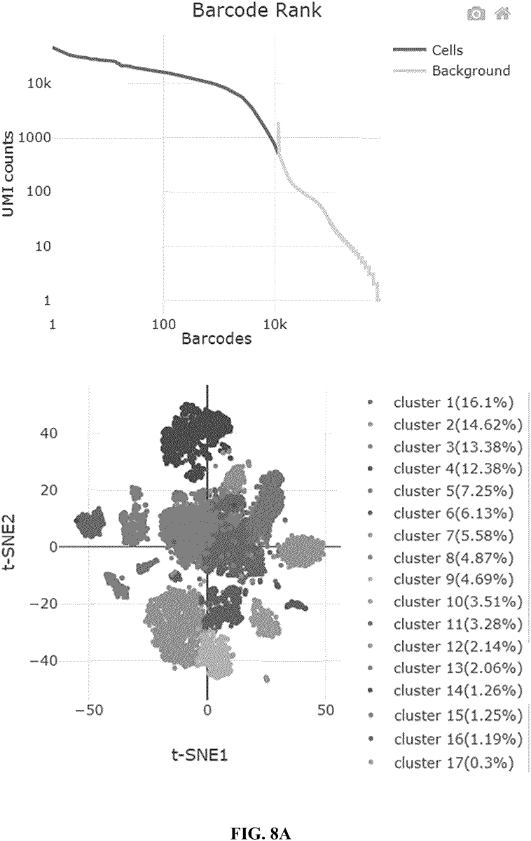

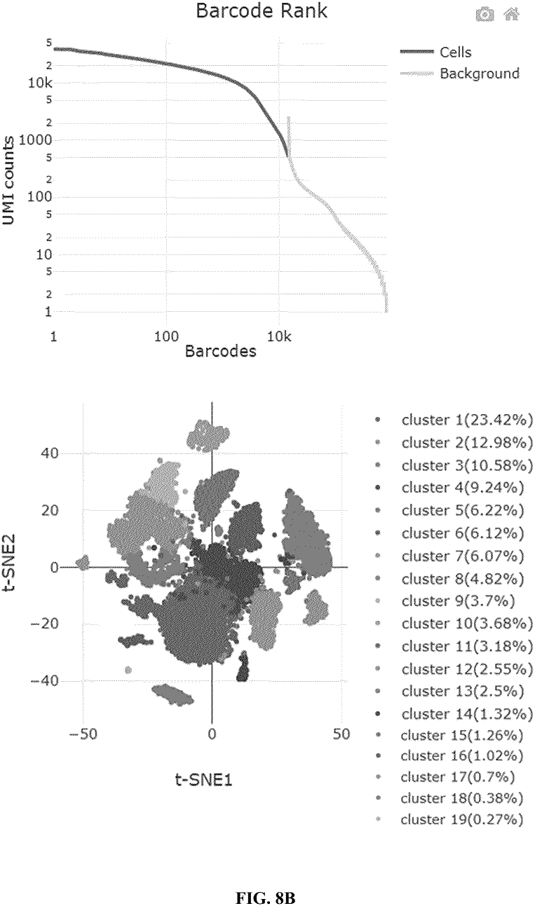

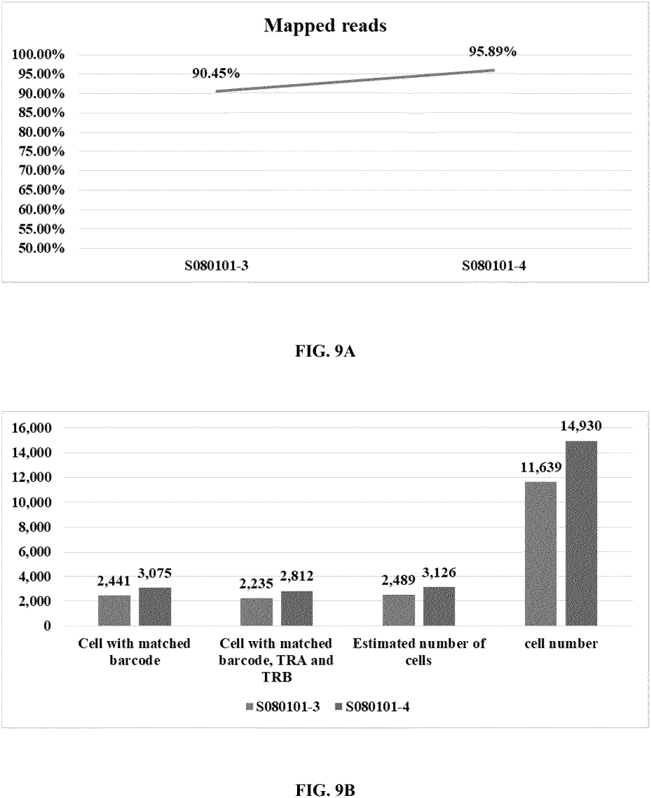

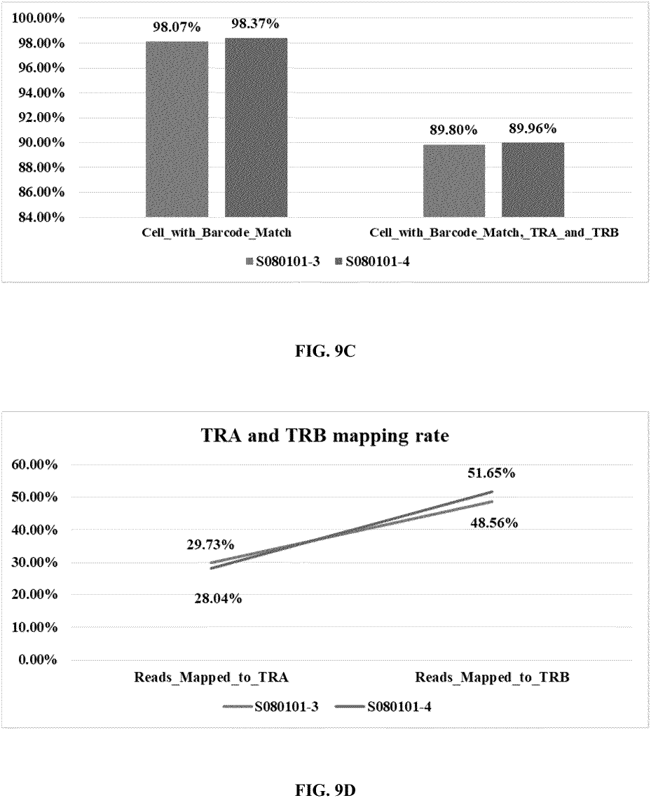

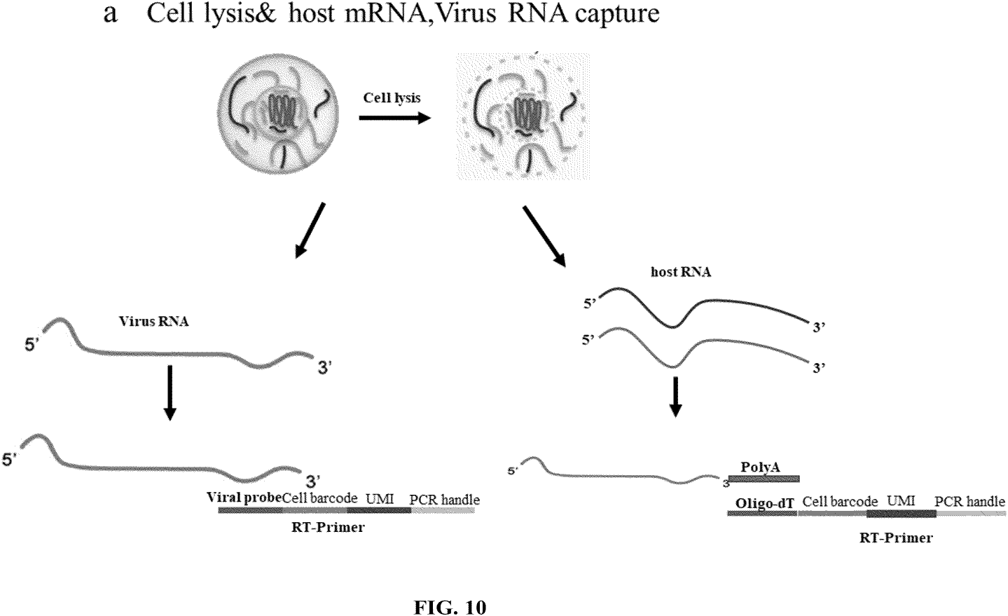

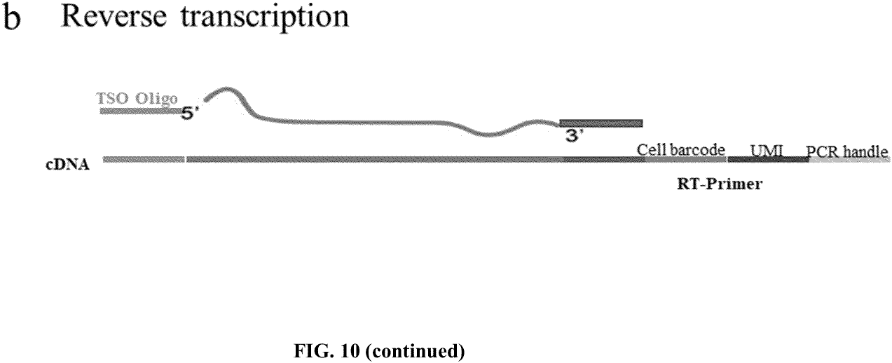

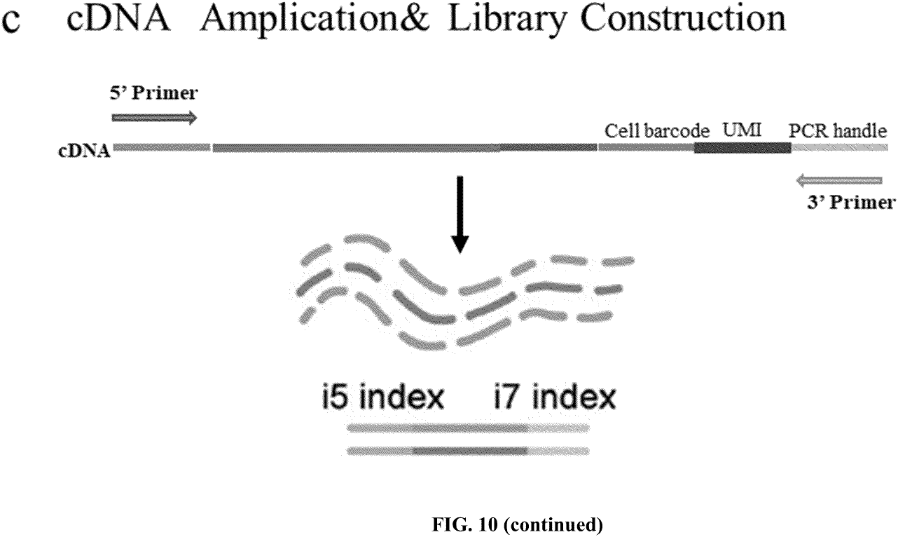

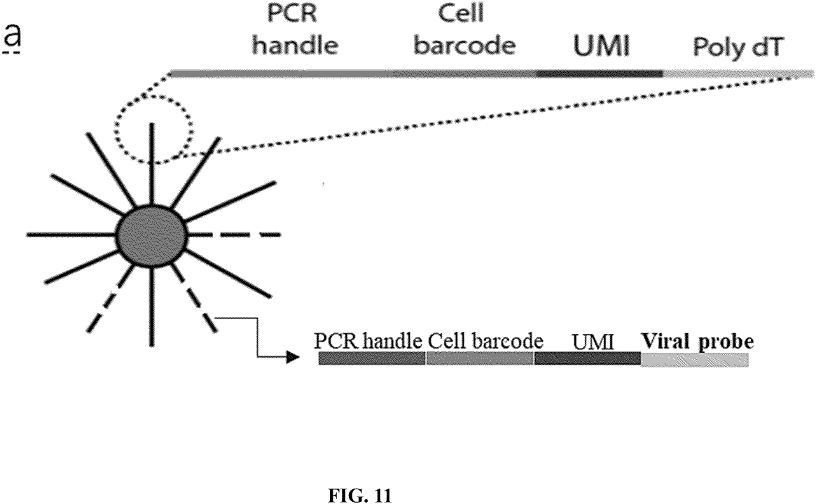

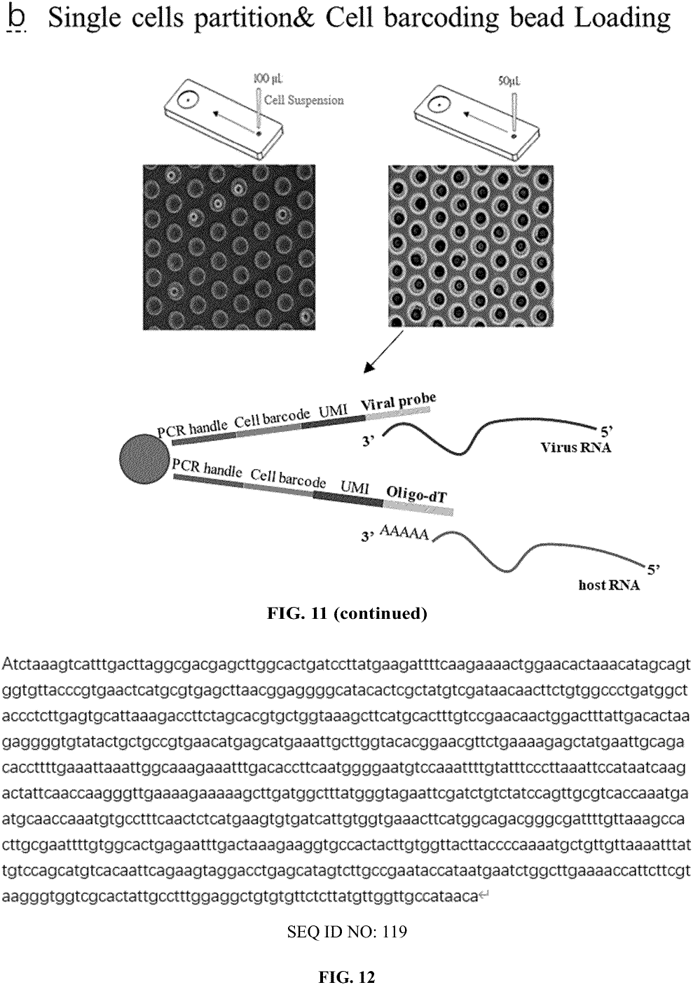

Procedures similar to what were used in Example 1 were used to analyze human oral cancer samples for TCR sequences. The results are shown in Table 9 FIGS. 7A-B, FIG. 8A (for S080101-1), FIG. 8B (for S080101-2), and FIGS. 9A-D.

TABLE 9

Results for human oral cancer samples

| | | | | | |
|---|---|---|---|---|---|
| Sample Name | Sample type | Cell viability | Cell concentration | Inputs | Name of TCR-enriched sample |
| S080101-1 | Human oral cancer sample-1 | 95% | 3 × 10⁵pcs/ml | 100 ul | S080101-3 |
| S080101-2 | Human oral | 93.5% | 3.2 × 10⁵ | 100 ul | S080101-4 |
| | cancer sample-2 | | pcs/ml | | |

TRA/TRB top 10 match clonetypes are shown in Table 10 (for S080101-3) and Table 11 (for S080101-4).

TABLE 10

TRA/TRB top 10 match clonetypes for S080101-3

| Clone type ID | aaSeqCDR3_TRA (SEQ ID NO) | aaSeqCDR3_TRB | Barcode count | percent |
|---|---|---|---|---|
| 1 | CVVKATSGTYKYIF (16) | CASSRLMGTRYNEQFF (17) | 151 | 6.19% |
| 2 | CAYRRAGQGAQKLVF (18) | CSARRMAPGPQYF (19) | 125 | 5.12% |
| 3 | CALLIGGTSYGKLTF (20) | CASSQTGNNNEQFF (21) | 45 | 1.84% |
| 4 | CATTGTGGFKTIF (22) | CASSQIRTGGFNEQFF (23) | 40 | 1.64% |
| 5 | CVMNGAGANNLFF (24) | CASSYSGTVTYEQYF (25) | 31 | 1.27% |
| 6 | CAVGGTSYGKLTF (26) | CSAPRRTDEQFF (27) | 28 | 1.15% |
| 7 | CALSEAL NTDKLIF (28) | CASSMNSGSYEQFF (29) | 23 | 0.94% |
| 8 | CAVRKSNNNDMRF (30) | CASSYPRTSGSPNYEQYF (31) | 22 | 0.9% |
| 9 | CAVLIGFGNVLHC (32) | CASSVAGGNEQFF (33) | 22 | 0.9% |
| 10 | CIVRARYLYNQGGKLIF (34) | CASSIRTGGENSPLHF (35) | 20 | 0.82% |

TABLE 11

TRA/TRB top 10 match clonetypes for S080101-4

| Clone type ID | aaSeqCDR3_TRA (SEQ ID NO) | aaSeqCDR3_TRB | Barcode count | percent |
|---|---|---|---|---|
| 1 | CVVKATSGTYKYIF (36) | CASSRLMGTRYNEQFF (37) | 186 | 6.05% |
| 2 | CAYRRAGQGAQKLVF (38) | CSARRMAPGPQYF (39) | 140 | 4.55% |
| 3 | CALLIGGTSYGKLTF (40) | CASSQTGNNNEQFF (41) | 71 | 2.31% |
| 4 | CVMNGAGANNLFF (42) | CASSYSGTVTYEQYF (43) | 48 | 1.56% |
| 6 | CAVGGTSYGKLTF (44) | CSAPRRTDEQFF (45) | 36 | 1.17% |
| 5 | CAVRLTGGFKTIF (46) | CASSLGQGGNTEAFF (47) | 35 | 1.14% |
| 7 | CATTGTGGFKTIF (48) | CASSQIRTGGFNEQFF (49) | 35 | 1.14% |
| 8 | N/A | CASSMNSGSYEQFF (50) | 32 | 1.04% |
| 9 | CAMRVRNFNKFYF (51) | CASSQERSGELETQYF (52) | 30 | 0.98% |
| 10 | CATEIQGAQKLVF (53) | CASSGGTSGILNTGELFF (54) | 30 | 0.98% |

### Example 3

### Detection of COVID-19 and Cell Sequences

In this example, to overcome the drawbacks of the current virus-host interactions analysis methods, probes binding to virus sequence were combined with oligo-dT to capture both host mRNA and virus nucleotide. The probes and oligo-dT contain the same PCR handle sequence, which can act as priming site for RT reactions and PCR amplification reactions.

As described herein, probes binding to virus sequence and oligo-dT can be added to the Magnetic capture beads, which enables capturing and reverse transcribing both mRNA and virus sequence. With unique cell barcodes in conjunction with the oligo-dT and probe sequence, cDNA molecules from the same single cell can be labeled and a group of single cells can be processed in parallel. The methods, compositions, kits and systems disclosed herein can be used to sequence and quantify the whole transcriptome of single cells together with the viral RNA from the same cell. By correlating gene expression with virus level in the same cell, several cellular functions involved in virus replication can be identified.

In this example, GEXSCOPE Single Cell RNAseq Library Construction kit (Singleron Biotechnologies) was used to show the technical feasibility and the utility of the methods, kits, compositions, and systems in high-throughput single cell virus-RNA sequencing. The experiment was conducted according to manufacturer’s instructions with modifications described below.

Cell barcoding Magnetic bead synthesis: cell barcoding magnetic beads were synthesized. The primers on all beads comprise a common sequence used for PCR amplification, a bead-specific cell barcode, a unique 8 molecular identifier (UMI), a oligo-dT sequence for capturing polyadenylated mRNAs and probe sequence annealing to COVID-19 sequence for capturing COVID-19 RNA. The sequence of the Probe is shown in Table 12.

TABLE 12

Probe sequences

| Probe | Sequence (5′ to 3′) | SEQ ID NO: |
|---|---|---|
| Round3-nCov1 | CGGCTACGAACGTGATCNNNNNNNNGCTCGTCGCCTAAGTCAA | 55 |
| Round3-nCov2 | CGGCTACGAACGTGATCNNNNNNNNATGTTCACGGCAGCAGTATA | 56 |
| Round3-nCov3 | CGGCTACGAACGTGATCNNNNNNNNGGTGACGCAACTGGATAGA | 57 |
| Round3-nCov4 | CGGCTACGAACGTGATCNNNNNNNNTATGGTATTCGGCAAGACTATG | 58 |
| Round3-nCov5 | CGGCTACGAACGTGATCNNNNNNNNGGCAACCAACATAAGAGAACA | 59 |

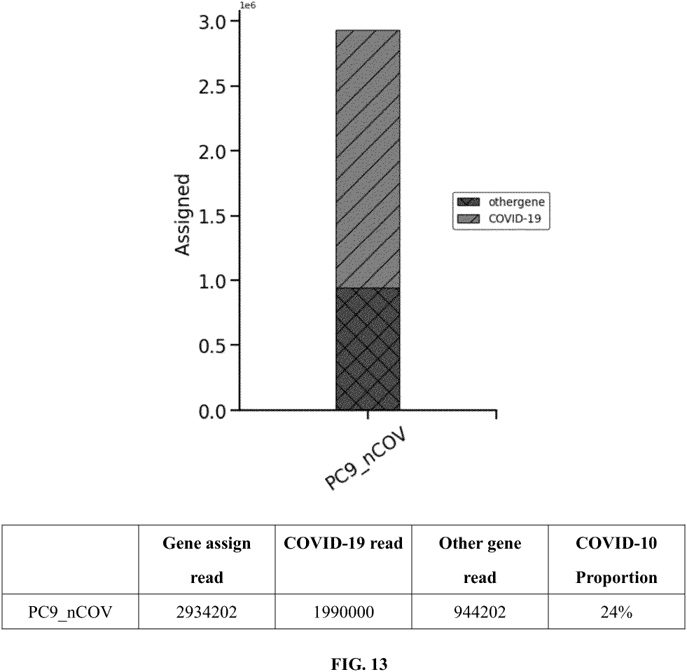

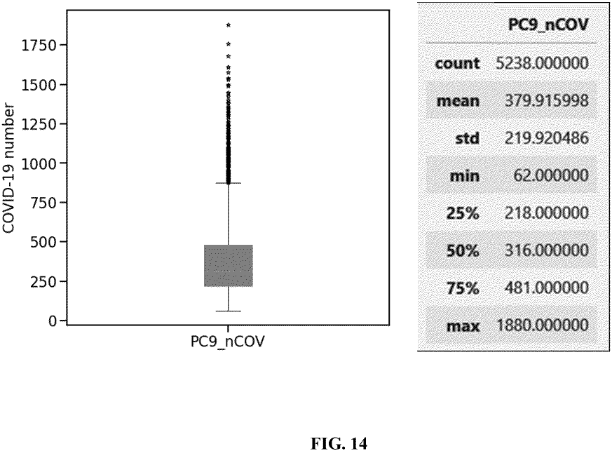

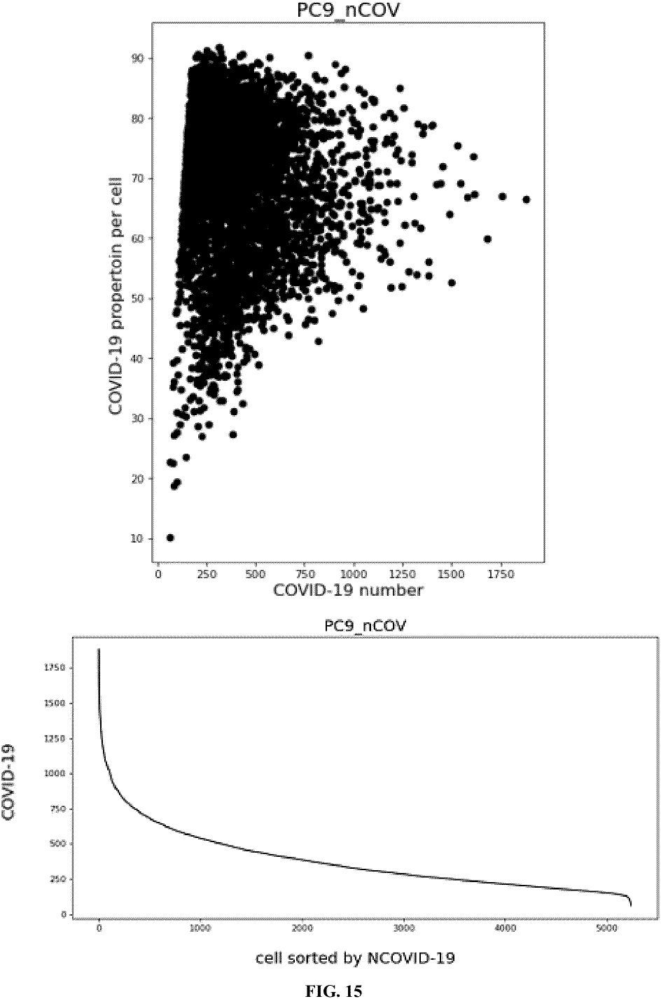

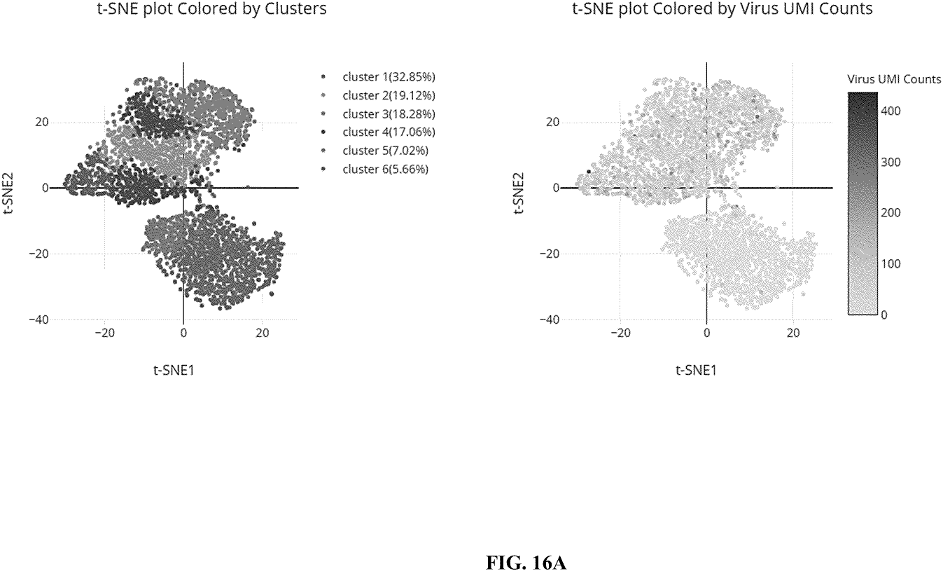

RNA of part of COVID-19 viral genome sequence (FIG. 12) was synthesized with in vitro transcription method. Single cell suspension of PC9 was first loaded onto the microchip to partition single cells into individual wells on the chip. Cell barcoding magnetic beads were then loaded to the microchip and washed. Only one bead can fall into each well on the microchip based on the diameters of the beads and well (about 25 um and 40 um, respectively). 100 ul cell lysis buffer which contains 10 ng COVID-19 RNA were then loaded into the chip and let incubate at room temperature for 20 minutes to lyse cells and capture RNAs. After 20 minutes, the magnetic beads, together with captured RNAs, were taken out of the microchip and subject to RT, template switching, cDNA amplification, and a part of cDNA was used to construct gene expression library using reagents from the GEXSCOPE kit and following manufacturer’s instructions. The resulting single cell RNAseq library was sequenced on Illumina NovaSeq with PE150 mode and analyzed with scopeTools bioinformatics workflow (Singleron Biotechnologies). FIG. 13 shows the detection of PC9 gene and COVID-19 gene at the same time. Cells can also be sorted based on the expression of COVID-19 (FIGS. 14 and 15).

### Example 4

### Detection of EBV Viral Sequences

Raji is a cell line containing EBV virus, and A549 is a negative control of a cell line that does not contain EBV virus. In order to test the reliability of the single-cell virus detection system, the following experiments are specially designed.

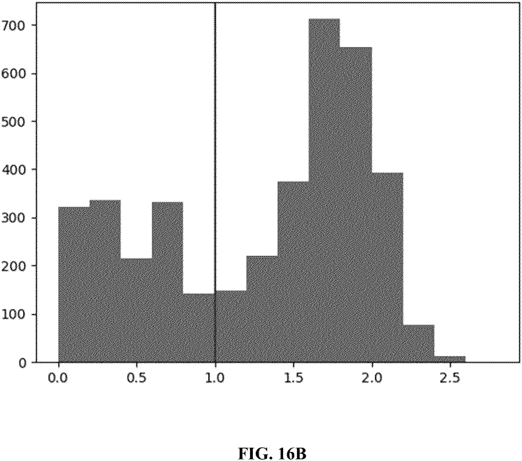

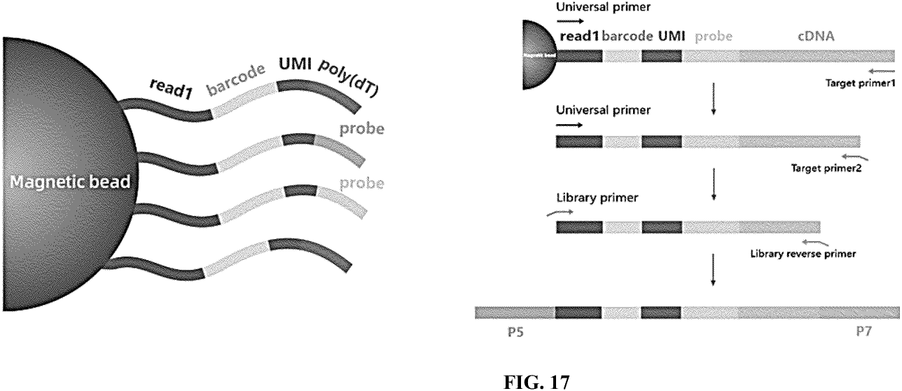

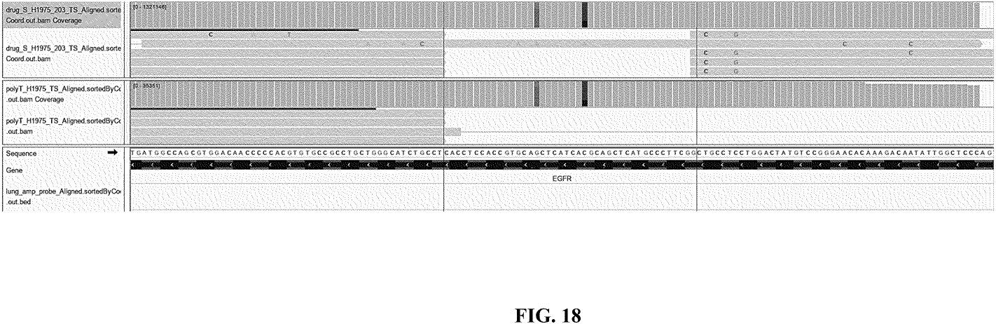

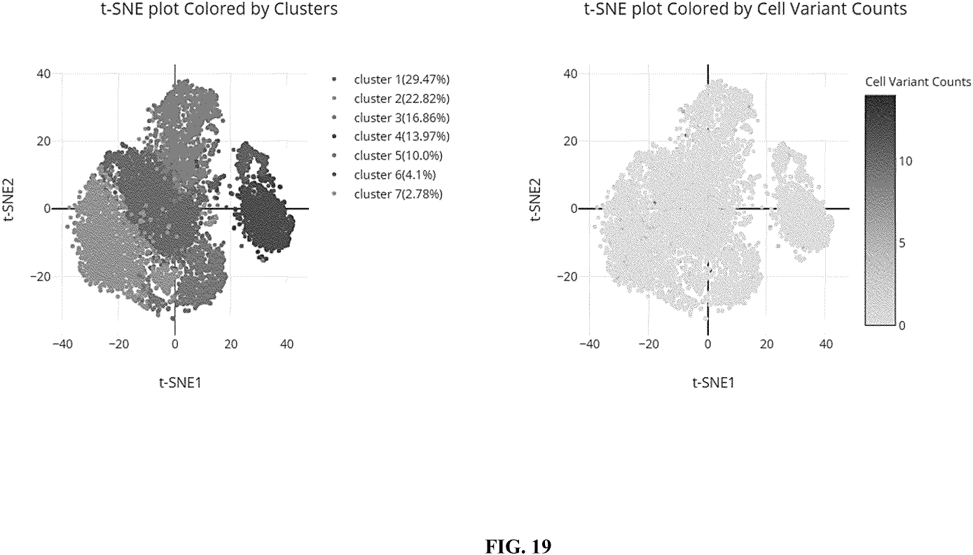

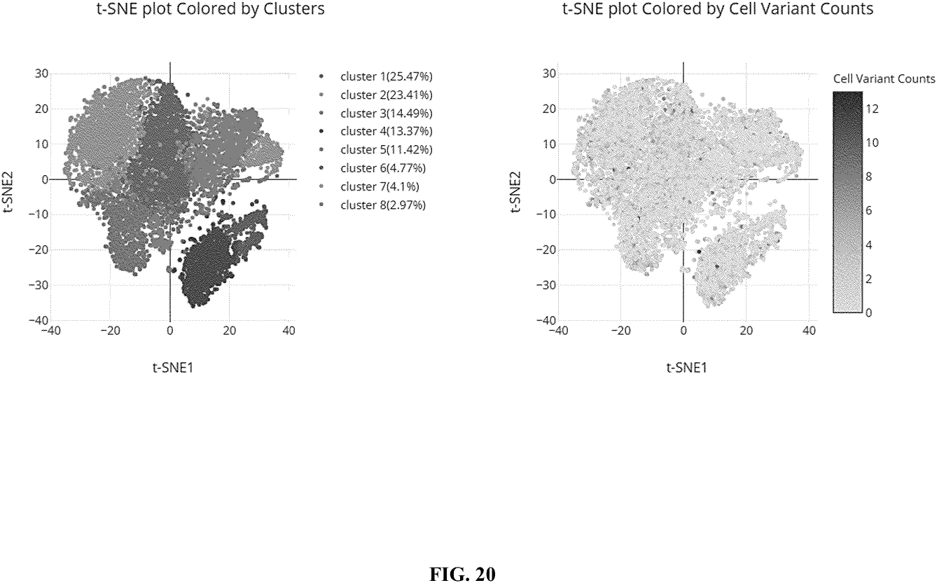

Raji and A549 cells were mixed in equal proportions. EBV virus capture magnetic beads were used to capture, construct transcriptome library and virus enrichment library. Celescope software was used to analyze, and the results are shown in FIGS. 16A-B and Table 13._

TABLE 13

Detection of EBV viral sequences

| Cell | cluster | EBV | No_EBV | Total | Rate of viral detection | Accuracy of detection |
|---|---|---|---|---|---|---|
| Raji | 2,3,4,6 | 2419 | 90 | 2509 | 96.4% | 94.2% |
| A549 | 1,5 | 150 | 1514 | 1664 | 9.0% | |
| | Total | 2569 | 1604 | 4173 | - | - |

### Example 5

### Detection of Single Cell Lung Cancer Druggable Mutations

In order to capture the transcriptome and the target region at the same time, a series of target region probe and the oligo-dT was designed in every capture magnetic beads. The capture probe and oligo-dT also contains the same PCR handle sequence, which can act as priming site for RT reactions and PCR amplification reactions. Probes binding to target regions, such as virus sequence, druggable sites and hotspots mutations of gene, can be used in the methods, kits, compositions and systems described herein.

As described herein, probe binding to lung cancer related hotspot mutation site can be attached to the magnetic capture beads. With unique cell barcodes in conjunction with the oligo-dT and probe sequence, cDNA molecules from the same single cell can be labeled and a group of single cells can be processed in parallel. The methods, compositions, kits and systems disclosed herein allows sequencing and quantifing the whole transcriptome of single cells together with the specific RNA from the same cell. By correlating gene expression with specific gene mutation information in the same cell, cells with gene mutations can be located.

GEXSCOPE Single Cell RNA-seq Library Construction kit (Singleron Biotechnologies) was used to show the technical feasibility and the utility of the methods, kits, compositions, and systems in high-throughput single cell target-RNA sequencing. The experiment was conducted according to manufacturer’s instructions with modifications described below.

Cell barcoding magnetic bead synthesis: cell barcoding magnetic beads were synthesized. The primers on all beads comprise a common sequence used for PCR amplification, a bead-specific cell barcode, a unique 12bp molecular identifier (UMI), and an oligo-dT sequence for capturing polyadenylated mRNAs and probe sequence annealing to target-gene sequence for capturing the interested gene.

The sequence of the probe is shown in Table 14:

TABLE 14

Probe sequences

| Probe | Sequence (5′ to 3′) | SEQ ID NO: |
|---|---|---|
| Round3EGFR-1: | CGGCTACGAACGTGATCNNNNNNNNGGTCCTCCAAGTAGTTCA | 60 |
| Round3EGFR-2: | CGGCTACGAACGTGATCNNNNNNNNCGACGGTCCTCCAAGTAG | 61 |
| Round3PIK3CA-1 | CGGCTACGAACGTGATCNNNNNNNNATTCCTATGCAATCGGTCTT | 62 |
| Round3PIK3CA-2 | CGGCTACGAACGTGATCNNNNNNNNAATGCTGTTCATGGATTGTG | 63 |
| Round3BRAF-1 | CGGCTACGAACGTGATCNNNNNNNNTGCCACTTTCCCTTGTAG | 64 |
| Round3-BRAF-2 | CGGCTACGAACGTGATCNNNNNNNNTCACTGCCACATCACCAT | 65 |
| Round3-RET-1 | CGGCTACGAACGTGATCNNNNNNNNATCTTGAAGGCATCCACG | 66 |
| Round3-RET-2 | CGGCTACGAACGTGATCNNNNNNNNTGAAGGCATCCACGGAGA | 67 |
| Round3-NTRK2-1 | CGGCTACGAACGTGATCNNNNNNNNTGAGGAACTTGTTGAGGTC | 68 |
| Round3-NTRK2-2 | CGGCTACGAACGTGATCNNNNNNNNGTGCCCTGAGGAACTTGT | 69 |
| Round3-ALK-1 | CGGCTACGAACGTGATCNNNNNNNNACAGCCTCCCTTTCTATAGTAG | 70 |
| Round3-ALK-2 | CGGCTACGAACGTGATCNNNNNNNNGCACAGCCTCCCTTTCTA | 71 |
| Round3-HARS-1 | CGGCTACGAACGTGATCNNNNNNNNGTACTGGTGGATGTCCTCAA | 72 |
| Round3-HARS-2 | CGGCTACGAACGTGATCNNNNNNNNGCCAGGTCACACTTGTTC | 73 |
| Round3-KRAS-1: | CGGCTACGAACGTGATCNNNNNNNNGGTACATCTTCAGAGTCCTTA | 74 |
| Round3-KRAS-2 | CGGCTACGAACGTGATCNNNNNNNNCCATAGGTACATCTTCAGAGT | 75 |
| Round3-NRAS-1: | CGGCTACGAACGTGATCNNNNNNNNGCTTAATCTGCTCCCTGTA | 76 |
| Round3-NRAS-2 | CGGCTACGAACGTGATCNNNNNNNNTAGGTACATCATCCGAGTCT | 77 |
| Round3-NTRK2-1 -1 | CGGCTACGAACGTGATCNNNNNNNNTATCACCTCATTGTTTGACAGC | 78 |
| Round3-NTRK2-2 -2 | CGGCTACGAACGTGATCNNNNNNNNCACTCTATCACCTCATTGTT | 79 |
| Round3-PIK3CA-5 | CGGCTACGAACGTGATCNNNNNNNNCAATAGTGTCTGTGACTCCAT | 80 |
| Round3-EGFR-3 | CGGCTACGAACGTGATCNNNNNNNNCAGAGTCCCTTATACACCGT | 81 |
| Round3-EGFR-4 | CGGCTACGAACGTGATCNNNNNNNNTCCAGAGTCCCTTATACACCG | 82 |
| Round3-EGFR-5 | CGGCTACGAACGTGATCNNNNNNNNTCCAATGCCATCCACTTG | 83 |
| Round3-EGFR-6 | CGGCTACGAACGTGATCNNNNNNNNACTTGATAGGCACTTTGCC | 84 |
| Round3-BRAF-8 | CGGCTACGAACGTGATCNNNNNNNNATGACTTCTGGTGCCATC | 85 |
| Round3-BRAF-9 | CGGCTACGAACGTGATCNNNNNNNNGCATTCTGATGACTTCTGG | 86 |

In this example, studies were performed to compare whether there are significant differences between the transcriptome indicators between druggable beads and polyT beads, and compare the SNP detection rate of the enriched library at the same time. The obtained results show that the method disclosed herein can significantly improve SNP detection efficiency.

NCI-H1975 cells (abbreviated herein as H1975) contains EGFR T790M mutation. Druggable beads and polyT beads, respectively, were used to analyze A549(with G12S mutation) lung cancer cells and H1975 lung cancer cells were analyzed based on the Singleron GEXSCOPE single cell RNA-sequencing kit. Single cell suspension of A549/H1975 was first loaded onto the microchip to partition single cells into individual wells on the chip. Cell barcoding magnetic beads were then loaded to the microchip. Only one bead could fall into each well on the microchip based on the diameters of the beads and well (about 25 µm and 40 µm, respectively). 100 µl cell lysis buffer were loaded into the chip and incubate at room temperature for 20 minutes to lyse cells and capture RNAs. After 20 minutes, the magnetic beads, together with captured RNAs, were taken out of the microchip and subject to RT, template switching, cDNA amplification, and a part of cDNA was used to construct gene expression library using reagents from the GEXSCOPE kit and following manufacturer’s instructions. The resulting single cell RNAseq library was sequenced on Illumina NovaSeq with PE150 mode and analyzed with Celescope Bioinformatics workflow (Singleron Biotechnologies).

Amplification primers were designed, and a targeted enrichment library was constructed for obtaining more sequence information of the interested regions at a lower sequencing depth. The same PCR handle was added to the 5 ‘end of all primers for next step PCR, target library was constructed using reagents from FocuSeqTM kit and following manufacturer’s instructions. The sequences are shown in Table 15. The results for detected mutations are shown in Table 16.

TABLE 15

Sequences

| Name | Sequences (5′ to 3′) | SEQ ID NO: |
|---|---|---|
| ALK-AMP-1 | ACTGGAGTTCAGACGTGTGCTCTTCCGATCTGCGGAAAAACATCACCCTCA | 87 |
| ALK-AMP-2 | ACTGGAGTTCAGACGTGTGCTCTTCCGATCTCTCATTCGGGGTCTGGGC | 88 |
| HRAS-AMP-1 | ACTGGAGTTCAGACGTGTGCTCTTCCGATCTTGTGAACGGTGGGGCAGGAG | 89 |
| HRAS-AMP-2 | ACTGGAGTTCAGACGTGTGCTCTTCCGATCTGTCGCGCCTGTGAACGGT | 90 |
| KRAS-AMP-1 | ACTGGAGTTCAGACGTGTGCTCTTCCGATCTTCAGCGGCTCCCAGGTGCGG | 91 |
| KRAS-AMP-2 | ACTGGAGTTCAGACGTGTGCTCTTCCGATCTCCAGGTGCGGGAGAGAGG | 92 |
| NRAS-AMP-1 | ACTGGAGTTCAGACGTGTGCTCTTCCGATCTGCAGTGGAGCTTGAGGTTC | 93 |
| NRAS-AMP-2 | ACTGGAGTTCAGACGTGTGCTCTTCCGATCTGTTCTTGCTGGTGTGAAATG | 94 |
| NTRK2-AMP-1 | ACTGGAGTTCAGACGTGTGCTCTTCCGATCTAGACACATTTGTTCAGCAC | 95 |
| NTRK2-AMP-2 | ACTGGAGTTCAGACGTGTGCTCTTCCGATCTTTCAGCACATCAAGCGAC | 96 |
| EGFR-AMP-1 | ACTGGAGTTCAGACGTGTGCTCTTCCGATCTCGGTGTATAAGGGACTCTGGA | 97 |
| EGFR-AMP-2 | ACTGGAGTTCAGACGTGTGCTCTTCCGATCTCCCAGAAGGTGAGAAAGTTA | 98 |
| PIK3CA-AMP-1 | ACTGGAGTTCAGACGTGTGCTCTTCCGATCTAGTAAAGGAGCCCAAGAATG | 99 |
| PIK3CA-AMP-2 | ACTGGAGTTCAGACGTGTGCTCTTCCGATCTAAAGGAGCCCAAGAATGC | 100 |
| BRAF-AMP-1 | ACTGGAGTTCAGACGTGTGCTCTTCCGATCTACGCCAAGTCAATCATCC | 101 |
| BRAF-AMP-2 | ACTGGAGTTCAGACGTGTGCTCTTCCGATCTTCAATCATCCACAGAGACC | 102 |
| RET-AMP-1 | ACTGGAGTTCAGACGTGTGCTCTTCCGATCTTGAGGAGGAGAAGTGCTT | 103 |
| RET-AMP-2 | ACTGGAGTTCAGACGTGTGCTCTTCCGATCTCCGAAGACATCCAGGATC | 104 |
| NTRK2-AMP-1 | ACTGGAGTTCAGACGTGTGCTCTTCCGATCTCACATCAAGCGACATAACAT | 105 |
| NTRK2-AMP-2 | ACTGGAGTTCAGACGTGTGCTCTTCCGATCTGCGACATAACATTGTTCTGA | 106 |
| ALK-AMP3 | ACTGGAGTTCAGACGTGTGCTCTTCCGATCTAAACATCACCCTCATTCGG | 107 |
| HRAS-AMP3 | ACTGGAGTTCAGACGTGTGCTCTTCCGATCTAGGAGACCCTGTAGGAGGA | 108 |
| KRAS-AMP3 | ACTGGAGTTCAGACGTGTGCTCTTCCGATCTGGGAGAGAGGCCTGCTGAAA | 109 |
| NTRK2-AMP3 | ACTGGAGTTCAGACGTGTGCTCTTCCGATCTCACATCAAGCGACATAACAT | 110 |

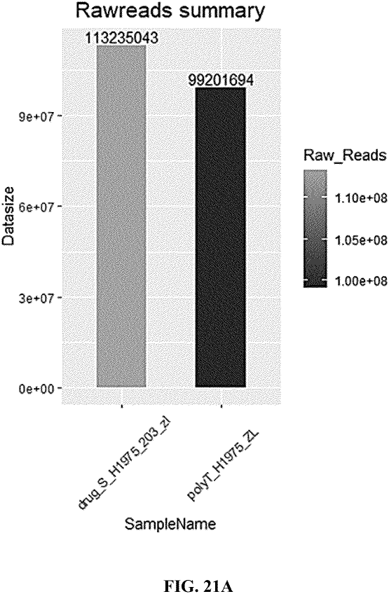

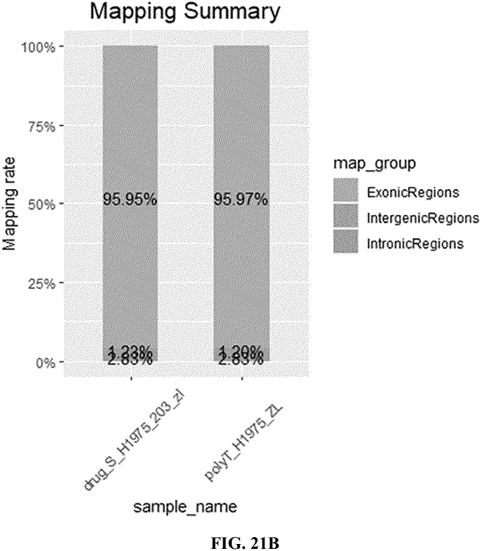

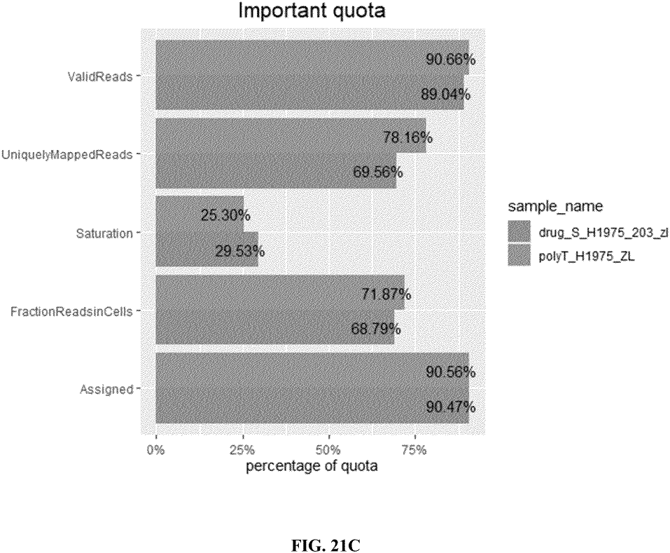

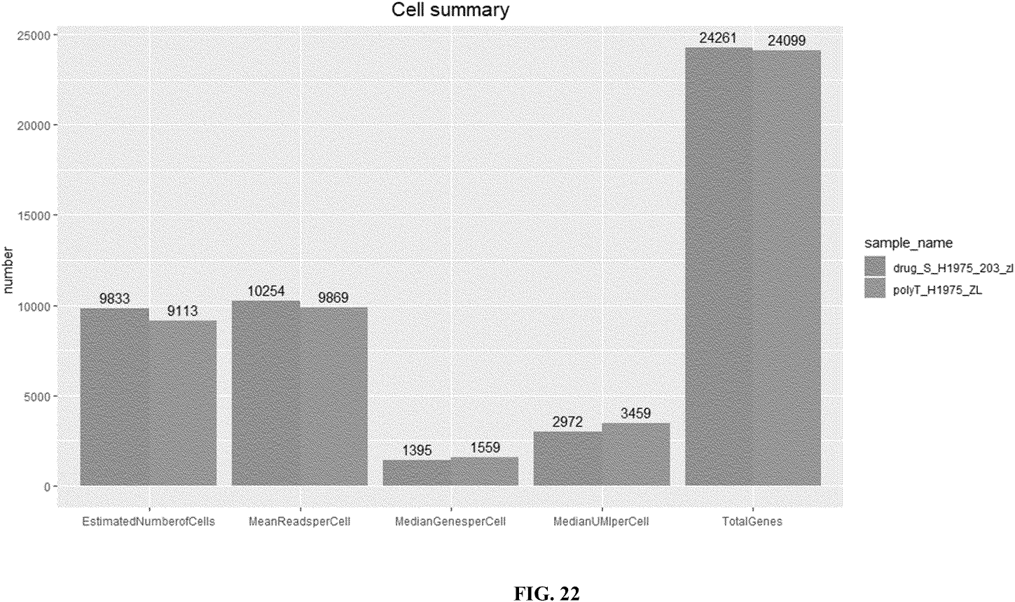

The results for comparison of transcription index between druggable beads and polyT beads are shown in FIGS. 21A-C and 22. The results indicate that in the case of similar sequencing depths, there is no obvious difference between the indicators of the transcriptome, which proves that the customized bead will not affect the data quality of the transcriptome. Obtained mutation statistics are shown in Table 16.

TABLE 16

Mutation statistics

| Sample Name | Cell Type | Bead | Cell Number | Number of cells with T790M mutation | Mutation detection rate |
|---|---|---|---|---|---|
| polyT_H1975_TS | NCI-H1975 | polyT | 9133 | 377 | 4.13% |
| drug_S_H1975_TS | NCI-H1975 | drug-S | 9833 | 6364 | 64.72% |

drug_S_H1975_TS: druggable beads capture H1975 cell lines polyT_H1975_TS: polyT beads capture H1975 cell line for control

### Example 6

### Detection of Lung Cancer Mutations

Cell line A549 contains the G12S mutation of the KRAS gene, and cell line U937 does not contain the G12S mutation. A549 and U937 cells were used in this example to determine the detection accuracy of the method described herein.

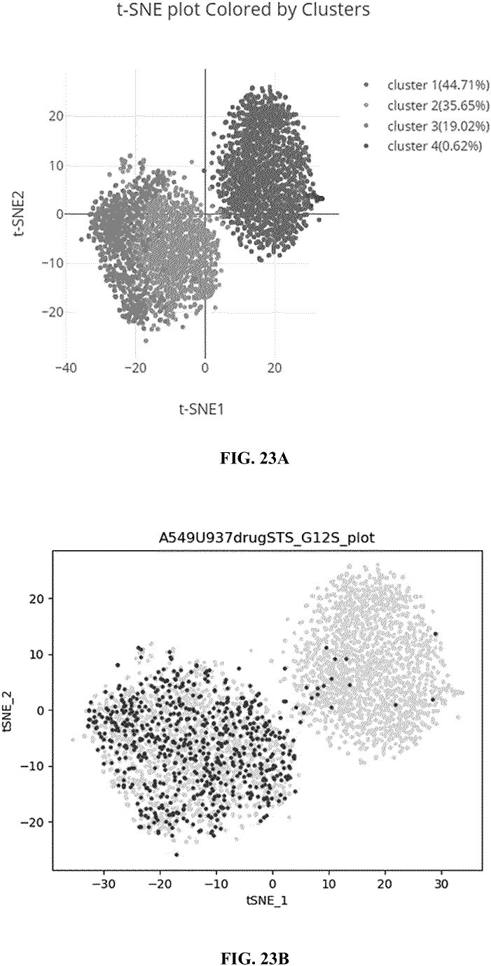

A549 and U937 cells were mixed in equal proportions, and captured with druggable beads. Transcriptome and enrichment library were constructed, tested and analyzed using Celescope SNP module. The results are shown in FIGS. 23A-B, and Table 13.

TABLE 13

Results from A549 and U937 cells

| Sample name | Cluster | Cell number | Cell type | Cell with G12S | Mutation detection accuracy |
|---|---|---|---|---|---|
| A549U937-dr | 1,4 | 2137 | U937 | 14 | 98% |
| ug-S | 2,3 | 2578 | A549 | 687 | |
| total | 1,2,3,4 | 4715 | - | 701 | - |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for single cell analysis comprising:
partitioning a cell and a bead attached with a plurality of barcode oligonucleotides into a partition, wherein each barcode oligonucleotide of the plurality of barcode oligonucleotides comprises a cell barcode and a unique molecular identifier (UMI), wherein first barcode oligonucleotides of the plurality of barcode oligonucleotides each comprises a poly-dT sequence capable of binding to a poly-A tail of a first messenger ribonucleic acid (mRNA) target, wherein second barcode oligonucleotides of the plurality of barcode oligonucleotides each comprises a poly-dT sequence and a probe sequence, and wherein the probe sequence is a non-poly-dT sequence and is capable of binding to a second RNA target at a sequence that is not a poly-A sequence;
hybridizing the first barcode oligonucleotides and the second barcode oligonucleotides attached to the bead in the partition with RNA targets associated with the cell in the partition;
reverse transcribing the RNA targets hybridized to the first barcode oligonucleotides and the second barcode oligonucleotides to generate barcoded complementary deoxyribonucleic acids (cDNAs);
amplifying the barcoded cDNAs; and
analyzing the amplified barcoded cDNAs, or products thereof.

2. The method of claim 1, wherein analyzing the amplified barcoded cDNAs comprises sequencing the amplified barcoded cDNAs to obtain sequencing information.

3. The method of claim 2, wherein analyzing the amplified barcoded cDNAs comprises:
determining an expression profile of each of one or more the RNA targets using a number of UMIs with different sequences associated with the RNA target in the sequencing information; and/or
determining an expression profile of the second RNA target using a number of UMIs with different sequences associated with the second RNA target in the sequencing information, optionally wherein the expression profile comprises an absolute abundance or a relative abundance.

4. The method of any one of claims 1-3, wherein analyzing the amplified barcoded cDNAs comprises:
determining a number of amplified barcoded cDNAs of each of one or more the RNA targets comprising UMIs with different sequences;
determining a number of amplified barcoded cDNAs the second RNA target comprising UMIs with different sequences; and/or
determining sequences of the amplified barcoded cDNAs of the second RNA target, or a portion thereof, comprising UMIs with different sequences.

5. A method for single cell sequencing comprising:
co-partitioning a plurality of cells and a plurality of beads into a plurality of partitions, wherein partitions of the plurality of partitions each comprises a single cell of the plurality of cells and a single bead of the plurality of beads, wherein each of the beads in the partitions of the plurality of partitions is attached with a plurality of barcode oligonucleotides, wherein each barcode oligonucleotide of the plurality of barcode oligonucleotides comprises (i) a cell barcode, (ii) a unique molecular identifier (UMI), and (iiia) a poly-dT sequence capable of binding to a poly-A region of a first nucleic acid target and/or (iiib) a probe sequence that is not a poly-dT sequence and is capable of binding to a second nucleic acid target;
barcoding nucleic acid targets associated with the cell in each partition of the partitions using first barcode oligonucleotides and second barcode oligonucleotides attached to the bead in the partition to generate barcoded nucleic acids; and
sequencing the barcoded nucleic acids, or products thereof, to obtain sequencing information.

6. A method for single cell sequencing comprising:
co-partitioning a plurality of cells and a plurality of beads into a plurality of partitions, wherein partitions of the plurality of partitions each comprises a single cell of the plurality of cells and a single bead of the plurality of beads, wherein each of the beads in the partitions of the plurality of partitions is attached with a plurality of barcode oligonucleotides, wherein each barcode oligonucleotide of the plurality of barcode oligonucleotides comprises (i) a cell barcode and (ii) a unique molecular identifier (UMI);
barcoding nucleic acid targets associated with the cell in each partition of the partitions to generate barcoded nucleic acids using (a) extension primers comprising a poly-dT sequence capable of binding to a poly-A region of a first nucleic acid target and/or a probe sequence that is not a poly-dT sequence and is capable of binding to a second nucleic acid target and (b) the first barcode oligonucleotides and the second barcode oligonucleotides attached to the bead in the partition as template switching oligonucleotides; and
sequencing the barcoded nucleic acids, or products thereof, to obtain sequencing information.

7. The method of any one of claims 5-6, wherein the nucleic acid targets comprise an ribonucleic acid (RNA), a messenger RNA (mRNA), and a deoxyribonucleic acid (DNA), and/or wherein the nucleic acid targets comprise nucleic acid targets of the cell, from the cell, in the cell, and/or on the surface of the cell.

8. The method of any one of claims 5-7, wherein barcoding the nucleic acids associated with the cell comprises:
hybridizing the first barcode oligonucleotides and the second barcode oligonucleotides attached to the bead in each partition of the partitions with nucleic acid targets associated with the cell in the partition;
extending the first barcode oligonucleotides and the second barcode oligonucleotides attached to the bead and hybridized to the nucleic acid targets using the nucleic acids as templates to generate single-stranded barcoded nucleic acids; and
generating double-stranded barcoded nucleic acids from the single-stranded barcoded nucleic acids.

9. The method of claim 8, wherein extending the single-stranded barcoded nucleic acids comprises further extending the single-stranded barcoded nucleic acids using a template switching oligonucleotide.

10. The method of any one of claims 8-9, comprising pooling the beads prior to extending the first barcode oligonucleotides and the second barcode oligonucleotides or prior to generating the double-stranded barcoded nucleic acids.

11. The method of any one of claims 8-9, wherein extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in bulk, and wherein generating the double-stranded barcoded nucleic acids comprises generating the double-stranded barcoded nucleic acids from the single-stranded barcoded nucleic acids in bulk.

12. The method of any one of claims 8-9, comprising pooling the beads subsequent to extending the first barcode oligonucleotides and the second barcode oligonucleotides attached to the bead to generate the single-stranded barcoded nucleic acids or subsequent to generating the double-stranded barcoded nucleic acids.

13. The method of any one of claims 8-9, wherein extending the first barcode oligonucleotides and the second barcode oligonucleotides attached to the bead and hybridized to the nucleic acid targets comprises extending the first barcode oligonucleotides and the second barcode oligonucleotides attached to the bead and hybridized to the nucleic acid targets in the partition, and wherein generating the double-stranded barcoded nucleic acids comprises generating the double-stranded barcoded nucleic acids from the single-stranded barcoded nucleic acids in the partition.

14. The method of any one of claims 5-13, comprising:
amplifying the barcoded nucleic acid to generate amplified barcoded nucleic acids, optionally wherein amplifying the barcoded nucleic acids comprises amplifying the barcoded nucleic acids using polymerase chain reaction (PCR) to generate the amplified barcoded nucleic acids;
processing the amplified barcoded nucleic acids to generate processed barcoded nucleic acids,
wherein sequencing the barcoded nucleic acids comprises sequencing the processed barcoded nucleic acids.

15. The method of claim 14, wherein processing the amplified barcoded nucleic acids comprises:
fragmenting the amplified barcoded nucleic acids to generate fragmented barcoded nucleic acids, optionally wherein fragmenting the amplified barcoded nucleic acids comprises fragmenting the amplified barcoded nucleic acids enzymatically to generate the fragmented barcoded nucleic acids;
adding a second polymerase chain reaction (PCR) primer-binding sequence, optionally wherein the second PCR primer-binding sequence comprises a Read 2 sequence; and
generating processed barcoded nucleic acids comprising sequencing primer sequences from the fragmented barcoded nucleic acids, optionally wherein the sequencing primer sequences comprise a P5 sequence and a P7 sequence.

16. The method of any one of claims 5-15, comprising analyzing the sequencing information.

17. The method of claim 16, wherein analyzing the sequencing information comprises:
determining an expression profile of each of one or more nucleic acid targets of the nucleic acid targets associated with the cell using a number of UMIs with different sequences associated with the nucleic acid target in the sequencing information;
determining an expression profile of the second nucleic acid target using a number of UMIs with different sequences associated with the second nucleic acid target in the sequencing information; and/or
determining sequences of the second nucleic acid target, or a portion thereof, associated with UMIs with different sequences,
optionally wherein the expression profile comprises an absolute abundance or a relative abundance, and
optionally wherein the expression profile comprises an RNA expression profile, an mRNA expression profile and/or a protein expression profile.

18. The method of any one of claims 5-17, wherein sequencing the barcoded nucleic acids, or products thereof, comprises sequencing products of the barcoded nucleic acids each comprising a P5 sequence, a Read 1 sequence, a cell barcode, a UMI, a poly-dT sequence, a probe sequence, a sequence of a nucleic acid target or a portion thereof, a Read 2 sequence, a sample index, and/or a P7 sequence to obtain sequencing information.

19. The method of any one of claims 1-18, wherein the partition is a droplet or a microwell.

20. The method of any one of claims 2-19, wherein the plurality of partitions comprises a plurality of microwells of a microwell array.

21. The method of any one of claims 2-20, wherein the plurality of partitions comprises at least 1000 partitions.

22. The method of any one of claims 2-21,
wherein at least 50% of partitions of the plurality of partitions comprise a single cell of the plurality of cells and a single bead of the plurality of beads,
wherein at most 10% of partitions of the plurality of partitions comprise two or more cells of the plurality of cells,
wherein at most 10% of partitions of the plurality of partitions comprise no cell of the plurality of cells,
wherein at most 10% of partitions of the plurality of partitions comprise two or more beads of the plurality of beads, and/or
wherein at most 10% of partitions of the plurality of partitions comprise no bead of the plurality of beads.

23. The method of any one of claims 1-22, wherein a length of the poly-dT sequence is at least 10 nucleotides in length, and/or wherein the probe sequence is at least 10 nucleotides in length.

24. The method of any one of claims 1-23, wherein first barcode oligonucleotides of the plurality of barcode oligonucleotides each comprises a poly-dT sequence capable of binding to a poly-A region of a first nucleic acid target.

25. The method of claim 24, wherein the poly-dT sequences of the first barcode oligonucleotides of the plurality of barcode oligonucleotides attached to a bead of the beads are identical, and/or wherein the poly-dT sequences of the first barcode oligonucleotides attached to the beads are identical.

26. The method of any one of claims 1-25, wherein second barcode oligonucleotides of the plurality of barcode oligonucleotides each comprises a probe sequence that is not a poly-dT sequence and is capable of binding to a second nucleic acid target.

27. The method of any one of claims 1-25, wherein second barcode oligonucleotides of the plurality of barcode oligonucleotides each comprises a poly-dT sequence and a probe sequence, and wherein the probe sequence is not a poly-dT sequence and is capable of binding to a second nucleic acid target.

28. The method of any one of claims 1-27, wherein second barcode oligonucleotides of the plurality of barcode oligonucleotides comprise probe sequences that are not poly-dT sequences and are capable of binding to an identical second nucleic acid target.

29. The method of any one of claims 26-28, wherein second barcode oligonucleotides of the plurality of barcode oligonucleotides comprise probe sequences that are not poly-dT sequences and are capable of binding to different second nucleic acid targets.

30. The method of any one of claims 1-29, wherein the probe sequences of barcode oligonucleotides of the plurality of barcode oligonucleotides comprise a degenerate sequence, optionally wherein a length of the degenerate sequence is at least 3, optionally wherein the degenerate sequence spans, or corresponds to, a mutation.

31. The method of any one of claims 1-30, wherein the probe sequences of barcode oligonucleotides of the plurality of barcode oligonucleotides span a region of interest.

32. The method of any one of claims 1-29, wherein the probe sequence is adjacent a region of interest.

33. The method of any one of claims 31-32, wherein the region of interest comprises a mutation, and/or wherein the region of interest comprises a variable region of a T-cell receptor (TCR), optionally wherein the TCR is TCR alpha or TCR beta.

34. The method of any one of claims 30-33, wherein the mutation comprises an insertion, a deletion, or a substitution, wherein the substitution comprises a single-nucleotide variant (SNV) or a single-nucleotide polymorphism (SNP), and/or wherein the mutation is related to a cancer.

35. The method of any one of claims 1-34, wherein the cell barcodes of two barcode oligonucleotides of the plurality of barcode oligonucleotides attached to a bead of the beads comprise an identical sequence, wherein the cell barcodes of two barcode oligonucleotides attached to two beads of the beads comprise different sequences, and/or wherein the cell barcode of each barcode oligonucleotide is at least 6 nucleotides in length.

36. The method of any one of claims 1-35, wherein the UMIs of two barcode oligonucleotides attached to a bead of the beads comprise different sequences, wherein the UMIs of two barcode oligonucleotides attached to two beads of the beads comprise an identical sequence, and/or wherein the UMI of each barcode oligonucleotide is at least 6 nucleotides in length.

37. The method of any one of claims 1-36, wherein each barcode oligonucleotide of the plurality of barcode oligonucleotides comprises a first polymerase chain reaction (PCR) primer-binding sequence, optionally wherein the first PCR primer-binding sequence comprises a Read 1 sequence.

38. The method of any one of claims 1-37, wherein barcode oligonucleotides of the plurality of barcode oligonucleotides are reversibly attached to, covalently attached to, or irreversibly attached to the bead.

39. The method of claim 38, wherein the bead is a gel bead, optionally wherein the gel bead is degradable upon application of a stimulus, optionally wherein the stimulus comprises a thermal stimulus, a chemical stimulus, a biological stimulus, a photo-stimulus, or a combination thereof.

40. The method of claim 38, wherein the bead is a solid bead and/or a magnetic bead.

41. The method of any one of claims 29-40, wherein the number of different second nucleic acid targets is at least 10.

42. The method of any one of claims 1-41, wherein the second nucleic acid target comprises a T-cell receptor (TCR), or an mRNA product thereof, wherein the probe sequence is capable of binding to a constant region, or a portion thereof, of the TCR, optionally wherein the TCR is TCR alpha or TCR beta.

43. The method of any one of claims 1-41, wherein the cell is a cancer cell, and wherein the second nucleic acid target is a cancer gene, or an mRNA product thereof.

44. The method of any one of claims 1-41, wherein the cell is infected with a virus, wherein the second nucleic acid target is a gene of the virus, or a nucleic acid product thereof, optionally wherein the virus is an RNA virus, optionally wherein the second nucleic acid target comprises an RNA of the gene of the virus, optionally thereby determining a transcriptomic profile of the cell and a RNA profile of the virus.

45. The method of any one of claims 1-44, wherein the second nucleic acid target comprises no poly-A tail and/or no poly-A region.

46. The method of any one of claims 1-44, wherein the second nucleic acid target comprises a poly-A region, optionally wherein the poly-A region is a poly-A tail.

47. The method of claim 46, wherein an abundance of molecules of the second nucleic acid target hybridized to, or barcoded using, the second barcode oligonucleotides is higher than an abundance of molecules of the second nucleic acid target hybridized to, or barcoded using, the first barcode oligonucleotides, thereby enriching the second nucleic acid target.

48. The method of claim 47, wherein the abundance of the molecules of the second nucleic acid target comprises a number of occurrences of the molecules of the second nucleic acid target.

49. The method of claim 47, wherein the abundance of the molecules of the second nucleic acid target comprises a number of occurrences of the molecules of the second nucleic acid target relative to a number of the first barcode oligonucleotides or a number of the second barcode oligonucleotides.

50. The method of any one of claims 5-49, comprising releasing the nucleic acids form the cell prior to barcoding the nucleic acid targets associated with the cell.

51. The method of any one of claims 5-50, comprising lysing the cell to release the nucleic acids form the cell.

52. The method of any one of claims 1-51, comprising enriching the one or more second nucleic acid targets using one or more enrichment primers.

53. The method of claim 52, wherein enriching the second nucleic acid targets comprises enriching the second nucleic acid targets using the enrichment primers of a panel, optionally wherein the panel is customizable.

54. A composition comprising a plurality of beads of any one of claims 1-53, wherein the cell barcodes of the plurality of barcode oligonucleotides attached to each of the plurality of beads are identical, and wherein the cell barcodes of barcodes oligonucleotide attached to different beads of the plurality of beads are different, optionally wherein the plurality of beads comprises at least 100 beads.

55. A kit comprising
a composition of claim 54; and
instructions of using the composition for single cell sequencing or analysis.

56. A method of generating beads comprising barcode oligonucleotides, the method comprising:
providing a plurality of beads each attached to a plurality of oligonucleotide barcodes, wherein each barcode oligonucleotide of the plurality of barcode oligonucleotides comprises a cell barcode, a unique molecular identifier (UMI), and a poly-dT sequence; and
adding a probe sequence that is non a poly-dT sequence and is capable of binding to a nucleic acid target, to 3′-end of each of barcode oligonucleotides of the plurality of barcode oligonucleotides.

57. The method of claim 56, wherein adding the probe sequence comprises adding the probe sequence to the 3′-end of each of the barcode oligonucleotides of the plurality of barcode oligonucleotides chemically.

58. The method of claim 56, wherein adding the probe sequence comprises adding the probe sequence to the 3′-end of each of the barcode oligonucleotides of the plurality of barcode oligonucleotides using an enzyme.

59. The method of claim 58, wherein the enzyme is a ligase, and wherein adding the probe sequence comprises ligating a probe oligonucleotide comprising the probe sequence to the 3′-end of each of the barcode oligonucleotides of the plurality of barcode oligonucleotides using the ligase.

60. The method of claim 58, wherein the enzyme is a DNA polymerase, and wherein adding the probe sequence comprises synthesizing the probe sequence at the 3′-end of each of the barcode oligonucleotides of the plurality of barcode oligonucleotides using the DNA polymerase.

61. A method of generating beads comprising barcode oligonucleotides, the method comprising:
providing a plurality of beads each attached to a plurality of oligonucleotide barcodes, wherein each barcode oligonucleotide of the plurality of barcode oligonucleotides comprises a cell barcode and a unique molecular identifier (UMI); and
adding to 3′-end of each of barcode oligonucleotides of the plurality of barcode oligonucleotides (i) a poly-dT sequence and/or (ii) a probe sequence that is a non-poly-dT sequence and is capable of binding to a nucleic acid target.


SEQUENCE LISTING (119 sequences)

SEQ ID NO: 1 - DNA, 20 bp, Artificial Sequence
  Synthetic Oligonucleotide
tgaaggcgtttgcacatgca

SEQ ID NO: 2 - DNA, 25 bp, Artificial Sequence
  Synthetic Oligonucleotide
tcaggcagtatctggagtcattgag

SEQ ID NO: 3 - DNA, 20 bp, Artificial Sequence
  Synthetic Oligonucleotide
agtctctcagctggtacacg

SEQ ID NO: 4 - DNA, 20 bp, Artificial Sequence
  Synthetic Oligonucleotide
tctgatggctcaaacacagc

SEQ ID NO: 5 - DNA, 24 bp, Artificial Sequence
  Synthetic Oligonucleotide
caaacgccttcaaaaaaaaaaaaa

SEQ ID NO: 6 - DNA, 24 bp, Artificial Sequence
  Synthetic Oligonucleotide
gatactgcctgaaaaaaaaaaaaa

SEQ ID NO: 7 - DNA, 24 bp, Artificial Sequence
  Synthetic Oligonucleotide
agctgagagactaaaaaaaaaaaa

SEQ ID NO: 8 - DNA, 24 bp, Artificial Sequence
  Synthetic Oligonucleotide
tgagccatcagaaaaaaaaaaaaa

SEQ ID NO: 9 - protein, 15 aa, Artificial Sequence
  TCR Sequence
CAFRDYGGSQGNLIF

SEQ ID NO: 10 - protein, 14 aa, Artificial Sequence
  TCR Sequence
CASSLAGAGNEQFF

SEQ ID NO: 11 - protein, 18 aa, Artificial Sequence
  TCR Sequence
CALIIGPGGGTSYGKLTF

SEQ ID NO: 12 - protein, 13 aa, Artificial Sequence
  TCR Sequence
CASSLNRVNEQYF

SEQ ID NO: 13 - protein, 13 aa, Artificial Sequence
  TCR Sequence
CASSIGIGREQYF

SEQ ID NO: 14 - protein, 14 aa, Artificial Sequence
  TCR Sequence
CAYAVGNTGNQFYF

SEQ ID NO: 15 - protein, 15 aa, Artificial Sequence
  TCR Sequence
CASSYMAGGLNEQFF

SEQ ID NO: 16 - protein, 13 aa, Artificial Sequence
  TCR Sequence
CSARQTSLYEQYF

SEQ ID NO: 17 - protein, 15 aa, Artificial Sequence
  TCR Sequence
CAFRDYGGSQGNLIF

SEQ ID NO: 18 - protein, 10 aa, Artificial Sequence
  TCR Sequence
CAGGSEKLVF

SEQ ID NO: 19 - protein, 16 aa, Artificial Sequence
  TCR Sequence
CASSLYQRGVEETQYF

SEQ ID NO: 20 - protein, 13 aa, Artificial Sequence
  TCR Sequence
CAAKTGGGNKLTF

SEQ ID NO: 21 - protein, 11 aa, Artificial Sequence
  TCR Sequence
CASSIFGEQYF

SEQ ID NO: 22 - protein, 11 aa, Artificial Sequence
  TCR Sequence
CAGHNTGKLIF

SEQ ID NO: 23 - protein, 12 aa, Artificial Sequence
  TCR Sequence
CASSLRLDEQYF

SEQ ID NO: 24 - protein, 14 aa, Artificial Sequence
  TCR Sequence
CVVKATSGTYKYIF

SEQ ID NO: 25 - protein, 16 aa, Artificial Sequence
  TCR Sequence
CASSRLMGTRYNEQFF

SEQ ID NO: 26 - protein, 15 aa, Artificial Sequence
  TCR Sequence
CAYRRAGQGAQKLVF

SEQ ID NO: 27 - protein, 13 aa, Artificial Sequence
  TCR Sequence
CSARRMAPGPQYF

SEQ ID NO: 28 - protein, 15 aa, Artificial Sequence
  TCR Sequence
CALLIGGTSYGKLTF

SEQ ID NO: 29 - protein, 14 aa, Artificial Sequence
  TCR Sequence
CASSQTGNNNEQFF

SEQ ID NO: 30 - protein, 13 aa, Artificial Sequence
  TCR Sequence
CATTGTGGFKTIF

SEQ ID NO: 31 - protein, 16 aa, Artificial Sequence
  TCR Sequence
CASSQIRTGGFNEQFF

SEQ ID NO: 32 - protein, 13 aa, Artificial Sequence
  TCR Sequence
CVMNGAGANNLFF

SEQ ID NO: 33 - protein, 15 aa, Artificial Sequence
  TCR Sequence
CASSYSGTVTYEQYF

SEQ ID NO: 34 - protein, 13 aa, Artificial Sequence
  TCR Sequence
CAVGGTSYGKLTF

SEQ ID NO: 35 - protein, 12 aa, Artificial Sequence
  TCR Sequence
CSAPRRTDEQFF

SEQ ID NO: 36 - protein, 15 aa, Artificial Sequence
  TCR Sequence
  misc_feature (8)..(8): Xaa can be any naturally occurring amino acid
CALSEALXNTDKLIF

SEQ ID NO: 37 - protein, 14 aa, Artificial Sequence
  TCR Sequence
CASSMNSGSYEQFF

SEQ ID NO: 38 - protein, 13 aa, Artificial Sequence
  TCR Sequence
CAVRKSNNNDMRF

SEQ ID NO: 39 - protein, 18 aa, Artificial Sequence
  TCR Sequence
CASSYPRTSGSPNYEQYF

SEQ ID NO: 40 - protein, 13 aa, Artificial Sequence
  TCR Sequence
CAVLIGFGNVLHC

SEQ ID NO: 41 - protein, 13 aa, Artificial Sequence
  TCR Sequence
CASSVAGGNEQFF

SEQ ID NO: 42 - protein, 17 aa, Artificial Sequence
  TCR Sequence
CIVRARYLYNQGGKLIF

SEQ ID NO: 43 - protein, 16 aa, Artificial Sequence
  TCR Sequence
CASSIRTGGENSPLHF

SEQ ID NO: 44 - protein, 14 aa, Artificial Sequence
  TCR Sequence
CVVKATSGTYKYIF

SEQ ID NO: 45 - protein, 16 aa, Artificial Sequence
  TCR Sequence
CASSRLMGTRYNEQFF

SEQ ID NO: 46 - protein, 15 aa, Artificial Sequence
  TCR Sequence
CAYRRAGQGAQKLVF

SEQ ID NO: 47 - protein, 13 aa, Artificial Sequence
  TCR Sequence
CSARRMAPGPQYF

SEQ ID NO: 48 - protein, 15 aa, Artificial Sequence
  TCR Sequence
CALLIGGTSYGKLTF

SEQ ID NO: 49 - protein, 14 aa, Artificial Sequence
  TCR Sequence
CASSQTGNNNEQFF

SEQ ID NO: 50 - protein, 13 aa, Artificial Sequence
  TCR Sequence
CVMNGAGANNLFF

SEQ ID NO: 51 - protein, 15 aa, Artificial Sequence
  TCR Sequence
CASSYSGTVTYEQYF

SEQ ID NO: 52 - protein, 13 aa, Artificial Sequence
  TCR Sequence
CAVGGTSYGKLTF

SEQ ID NO: 53 - protein, 12 aa, Artificial Sequence
  TCR Sequence
CSAPRRTDEQFF

SEQ ID NO: 54 - protein, 13 aa, Artificial Sequence
  TCR Sequence
CAVRLTGGFKTIF

SEQ ID NO: 55 - protein, 15 aa, Artificial Sequence
  TCR Sequence
CASSLGQGGNTEAFF

SEQ ID NO: 56 - protein, 13 aa, Artificial Sequence
  TCR Sequence
CATTGTGGFKTIF

SEQ ID NO: 57 - protein, 16 aa, Artificial Sequence
  TCR Sequence
CASSQIRTGGFNEQFF

SEQ ID NO: 58 - protein, 14 aa, Artificial Sequence
  TCR Sequence
CASSMNSGSYEQFF

SEQ ID NO: 59 - protein, 13 aa, Artificial Sequence
  TCR Sequence
CAMRVRNFNKFYF

SEQ ID NO: 60 - protein, 16 aa, Artificial Sequence
  TCR Sequence
CASSQERSGELETQYF

SEQ ID NO: 61 - protein, 13 aa, Artificial Sequence
  TCR Sequence
CATEIQGAQKLVF

SEQ ID NO: 62 - protein, 18 aa, Artificial Sequence
  TCR Sequence
CASSGGTSGILNTGELFF

SEQ ID NO: 63 - DNA, 43 bp, Artificial Sequence
  Synthetic Oligonucleotide
  misc_feature (18)..(25): n is a, c, g, or t
cggctacgaacgtgatcnnnnnnnngctcgtcgcctaagtcaa

SEQ ID NO: 64 - DNA, 45 bp, Artificial Sequence
  Synthetic Oligonucleotide
  misc_feature (18)..(25): n is a, c, g, or t
cggctacgaacgtgatcnnnnnnnnatgttcacggcagcagtata

SEQ ID NO: 65 - DNA, 44 bp, Artificial Sequence
  Synthetic Oligonucleotide
  misc_feature (18)..(25): n is a, c, g, or t
cggctacgaacgtgatcnnnnnnnnggtgacgcaactggataga

SEQ ID NO: 66 - DNA, 47 bp, Artificial Sequence
  Synthetic Oligonucleotide
  misc_feature (18)..(25): n is a, c, g, or t
cggctacgaacgtgatcnnnnnnnntatggtattcggcaagactatg

SEQ ID NO: 67 - DNA, 46 bp, Artificial Sequence
  Synthetic Oligonucleotide
  misc_feature (18)..(25): n is a, c, g, or t
cggctacgaacgtgatcnnnnnnnnggcaaccaacataagagaaca

SEQ ID NO: 68 - DNA, 43 bp, Artificial Sequence
  Synthetic Oligonucleotide
  misc_feature (18)..(25): n is a, c, g, or t
cggctacgaacgtgatcnnnnnnnnggtcctccaagtagttca

SEQ ID NO: 69 - DNA, 43 bp, Artificial Sequence
  Synthetic Oligonucleotide
  misc_feature (18)..(25): n is a, c, g, or t
cggctacgaacgtgatcnnnnnnnncgacggtcctccaagtag

SEQ ID NO: 70 - DNA, 45 bp, Artificial Sequence
  Synthetic Oligonucleotide
  misc_feature (18)..(25): n is a, c, g, or t
cggctacgaacgtgatcnnnnnnnnattcctatgcaatcggtctt

SEQ ID NO: 71 - DNA, 45 bp, Artificial Sequence
  Synthetic Oligonucleotide
  misc_feature (18)..(25): n is a, c, g, or t
cggctacgaacgtgatcnnnnnnnnaatgctgttcatggattgtg

SEQ ID NO: 72 - DNA, 43 bp, Artificial Sequence
  Synthetic Oligonucleotide
  misc_feature (18)..(25): n is a, c, g, or t
cggctacgaacgtgatcnnnnnnnntgccactttcccttgtag

SEQ ID NO: 73 - DNA, 43 bp, Artificial Sequence
  Synthetic Oligonucleotide
  misc_feature (18)..(25): n is a, c, g, or t
cggctacgaacgtgatcnnnnnnnntcactgccacatcaccat

SEQ ID NO: 74 - DNA, 43 bp, Artificial Sequence
  Synthetic Oligonucleotide
  misc_feature (18)..(25): n is a, c, g, or t
cggctacgaacgtgatcnnnnnnnnatcttgaaggcatccacg

SEQ ID NO: 75 - DNA, 43 bp, Artificial Sequence
  Synthetic Oligonucleotide
  misc_feature (18)..(25): n is a, c, g, or t
cggctacgaacgtgatcnnnnnnnntgaaggcatccacggaga

SEQ ID NO: 76 - DNA, 44 bp, Artificial Sequence
  Synthetic Oligonucleotide
  misc_feature (18)..(25): n is a, c, g, or t
cggctacgaacgtgatcnnnnnnnntgaggaacttgttgaggtc

SEQ ID NO: 77 - DNA, 43 bp, Artificial Sequence
  Synthetic Oligonucleotide
  misc_feature (18)..(25): n is a, c, g, or t
cggctacgaacgtgatcnnnnnnnngtgccctgaggaacttgt

SEQ ID NO: 78 - DNA, 47 bp, Artificial Sequence
  Synthetic Oligonucleotide
  misc_feature (18)..(25): n is a, c, g, or t
cggctacgaacgtgatcnnnnnnnnacagcctccctttctatagtag

SEQ ID NO: 79 - DNA, 43 bp, Artificial Sequence
  Synthetic Oligonucleotide
  misc_feature (18)..(25): n is a, c, g, or t
cggctacgaacgtgatcnnnnnnnngcacagcctccctttcta

SEQ ID NO: 80 - DNA, 45 bp, Artificial Sequence
  Synthetic Oligonucleotide
  misc_feature (18)..(25): n is a, c, g, or t
cggctacgaacgtgatcnnnnnnnngtactggtggatgtcctcaa

SEQ ID NO: 81 - DNA, 43 bp, Artificial Sequence
  Synthetic Oligonucleotide
  misc_feature (18)..(25): n is a, c, g, or t
cggctacgaacgtgatcnnnnnnnngccaggtcacacttgttc

SEQ ID NO: 82 - DNA, 46 bp, Artificial Sequence
  Synthetic Oligonucleotide
  misc_feature (18)..(25): n is a, c, g, or t
cggctacgaacgtgatcnnnnnnnnggtacatcttcagagtcctta

SEQ ID NO: 83 - DNA, 46 bp, Artificial Sequence
  Synthetic Oligonucleotide
  misc_feature (18)..(25): n is a, c, g, or t
cggctacgaacgtgatcnnnnnnnnccataggtacatcttcagagt

SEQ ID NO: 84 - DNA, 44 bp, Artificial Sequence
  Synthetic Oligonucleotide
  misc_feature (18)..(25): n is a, c, g, or t
cggctacgaacgtgatcnnnnnnnngcttaatctgctccctgta

SEQ ID NO: 85 - DNA, 45 bp, Artificial Sequence
  Synthetic Oligonucleotide
  misc_feature (18)..(25): n is a, c, g, or t
cggctacgaacgtgatcnnnnnnnntaggtacatcatccgagtct

SEQ ID NO: 86 - DNA, 47 bp, Artificial Sequence
  Synthetic Oligonucleotide
  misc_feature (18)..(25): n is a, c, g, or t
cggctacgaacgtgatcnnnnnnnntatcacctcattgtttgacagc

SEQ ID NO: 87 - DNA, 45 bp, Artificial Sequence
  Synthetic Oligonucleotide
  misc_feature (18)..(25): n is a, c, g, or t
cggctacgaacgtgatcnnnnnnnncactctatcacctcattgtt

SEQ ID NO: 88 - DNA, 46 bp, Artificial Sequence
  Synthetic Oligonucleotide
  misc_feature (18)..(25): n is a, c, g, or t
cggctacgaacgtgatcnnnnnnnncaatagtgtctgtgactccat

SEQ ID NO: 89 - DNA, 45 bp, Artificial Sequence
  Synthetic Oligonucleotide
  misc_feature (18)..(25): n is a, c, g, or t
cggctacgaacgtgatcnnnnnnnncagagtcccttatacaccgt

SEQ ID NO: 90 - DNA, 46 bp, Artificial Sequence
  Synthetic Oligonucleotide
  misc_feature (18)..(25): n is a, c, g, or t
cggctacgaacgtgatcnnnnnnnntccagagtcccttatacaccg

SEQ ID NO: 91 - DNA, 43 bp, Artificial Sequence
  Synthetic Oligonucleotide
  misc_feature (18)..(25): n is a, c, g, or t
cggctacgaacgtgatcnnnnnnnntccaatgccatccacttg

SEQ ID NO: 92 - DNA, 44 bp, Artificial Sequence
  Synthetic Oligonucleotide
  misc_feature (18)..(25): n is a, c, g, or t
cggctacgaacgtgatcnnnnnnnnacttgataggcactttgcc

SEQ ID NO: 93 - DNA, 43 bp, Artificial Sequence
  Synthetic Oligonucleotide
  misc_feature (18)..(25): n is a, c, g, or t
cggctacgaacgtgatcnnnnnnnnatgacttctggtgccatc

SEQ ID NO: 94 - DNA, 44 bp, Artificial Sequence
  Synthetic Oligonucleotide
  misc_feature (18)..(25): n is a, c, g, or t
cggctacgaacgtgatcnnnnnnnngcattctgatgacttctgg

SEQ ID NO: 95 - DNA, 51 bp, Artificial Sequence
  Synthetic Oligonucleotide
actggagttcagacgtgtgctcttccgatctgcggaaaaacatcaccctca

SEQ ID NO: 96 - DNA, 49 bp, Artificial Sequence
  Synthetic Oligonucleotide
actggagttcagacgtgtgctcttccgatctctcattcggggtctgggc

SEQ ID NO: 97 - DNA, 51 bp, Artificial Sequence
  Synthetic Oligonucleotide
actggagttcagacgtgtgctcttccgatcttgtgaacggtggggcaggag

SEQ ID NO: 98 - DNA, 49 bp, Artificial Sequence
  Synthetic Oligonucleotide
actggagttcagacgtgtgctcttccgatctgtcgcgcctgtgaacggt

SEQ ID NO: 99 - DNA, 51 bp, Artificial Sequence
  Synthetic Oligonucleotide
actggagttcagacgtgtgctcttccgatcttcagcggctcccaggtgcgg

SEQ ID NO: 100 - DNA, 49 bp, Artificial Sequence
  Synthetic Oligonucleotide
actggagttcagacgtgtgctcttccgatctccaggtgcgggagagagg

SEQ ID NO: 101 - DNA, 50 bp, Artificial Sequence
  Synthetic Oligonucleotide
actggagttcagacgtgtgctcttccgatctgcagtggagcttgaggttc

SEQ ID NO: 102 - DNA, 51 bp, Artificial Sequence
  Synthetic Oligonucleotide
actggagttcagacgtgtgctcttccgatctgttcttgctggtgtgaaatg

SEQ ID NO: 103 - DNA, 50 bp, Artificial Sequence
  Synthetic Oligonucleotide
actggagttcagacgtgtgctcttccgatctagacacatttgttcagcac

SEQ ID NO: 104 - DNA, 49 bp, Artificial Sequence
  Synthetic Oligonucleotide
actggagttcagacgtgtgctcttccgatctttcagcacatcaagcgac

SEQ ID NO: 105 - DNA, 52 bp, Artificial Sequence
  Synthetic Oligonucleotide
actggagttcagacgtgtgctcttccgatctcggtgtataagggactctgga

SEQ ID NO: 106 - DNA, 51 bp, Artificial Sequence
  Synthetic Oligonucleotide
actggagttcagacgtgtgctcttccgatctcccagaaggtgagaaagtta

SEQ ID NO: 107 - DNA, 51 bp, Artificial Sequence
  Synthetic Oligonucleotide
actggagttcagacgtgtgctcttccgatctagtaaaggagcccaagaatg

SEQ ID NO: 108 - DNA, 49 bp, Artificial Sequence
  Synthetic Oligonucleotide
actggagttcagacgtgtgctcttccgatctaaaggagcccaagaatgc

SEQ ID NO: 109 - DNA, 49 bp, Artificial Sequence
  Synthetic Oligonucleotide
actggagttcagacgtgtgctcttccgatctacgccaagtcaatcatcc

SEQ ID NO: 110 - DNA, 50 bp, Artificial Sequence
  Synthetic Oligonucleotide
actggagttcagacgtgtgctcttccgatcttcaatcatccacagagacc

SEQ ID NO: 111 - DNA, 49 bp, Artificial Sequence
  Synthetic Oligonucleotide
actggagttcagacgtgtgctcttccgatcttgaggaggagaagtgctt

SEQ ID NO: 112 - DNA, 49 bp, Artificial Sequence
  Synthetic Oligonucleotide
actggagttcagacgtgtgctcttccgatctccgaagacatccaggatc

SEQ ID NO: 113 - DNA, 51 bp, Artificial Sequence
  Synthetic Oligonucleotide
actggagttcagacgtgtgctcttccgatctcacatcaagcgacataacat

SEQ ID NO: 114 - DNA, 51 bp, Artificial Sequence
  Synthetic Oligonucleotide
actggagttcagacgtgtgctcttccgatctgcgacataacattgttctga

SEQ ID NO: 115 - DNA, 50 bp, Artificial Sequence
  Synthetic Oligonucleotide
actggagttcagacgtgtgctcttccgatctaaacatcaccctcattcgg

SEQ ID NO: 116 - DNA, 50 bp, Artificial Sequence
  Synthetic Oligonucleotide
actggagttcagacgtgtgctcttccgatctaggagaccctgtaggagga

SEQ ID NO: 117 - DNA, 51 bp, Artificial Sequence
  Synthetic Oligonucleotide
actggagttcagacgtgtgctcttccgatctgggagagaggcctgctgaaa

SEQ ID NO: 118 - DNA, 51 bp, Artificial Sequence; identical to SEQ ID NO: 113
  Synthetic Oligonucleotide

SEQ ID NO: 119 - DNA, 840 bp, Artificial Sequence
  Synthetic Oligonucleotide
atctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataaca